(19) United States
(12) Patent Application Publication
(10) Pub. No.: US 2005/0239061 A1
(43) Pub. Date: Oct. 27, 2005

(54) Identification and use of effectors and allosteric molecules for the alteration of gene expression

(76) Inventors: William S. Marshall, Boulder, CO (US); Anastasia Khvorova, Boulder, CO (US); Sumedha Jayasena, Thousand Oaks, CA (US)

Correspondence Address: AMGEN INC., MAIL STOP 28-2-C, ONE AMGEN CENTER DRIVE, THOUSAND OAKS, CA, 91320-1799, US

(21) Appl. No.: 10/220,403

(22) PCT Filed: Mar. 1, 2001
(86) PCT No.: PCT/US01/06615

§ 371 Date: Feb. 26, 2003

Related U.S. Application Data
(63) Continuation-in-part of application No. 09/729,258, filed on Nov. 28, 2000, now abandoned.
(60) Provisional application No. 60/186,248, filed on Mar. 1, 2000.
(60) Provisional application No. 60/186,248, filed on Mar. 1, 2000.

(51) Int. Cl. C12Q 1/68; G01N 33/53; G06F 19/00; G01N 33/48; G01N 33/50

(52) U.S. Cl. 435/6; 435/69.1; 435/7.1; 702/19

(57) ABSTRACT

The present invention relates to the construction of an allosteric control module in which a catalytic RNA forms a part of or is linked to an effector-binding RNA domain or aptamer. These constructs place the activity of the catalytic RNA under the control of the effector and require the presence of an appropriate effector for activation or inactivation. The present invention provides means to identify useful effector molecules as well as their use to evolve cognate aptamers. The invention involves both the evolution of RNA sequences which bind the effector and a selection proces in which the allosteric control modules are identified by their catalytic function in the presence and absence of the effector. The resulting regulatable catalytic RNAs may be used to alter the expression of a target RNA molecule in a controlled fashion.

### FIELD OF THE INVENTION

The present invention relates to the use of identified effectors to alter the catalytic activity of polynucleotides and to methods of use of these components in the alteration of gene expression.

### BACKGROUND OF THE INVENTION

The following is a brief description of enzymatic nucleic acid molecules. This summary is not meant to be all-inclusive but is provided only for a better understanding of the invention that follows. This summary is not an admission that all of the work described below is prior art to the claimed invention.

Enzymatic nucleic acid molecules (e.g., ribozymes) are nucleic acids capable of catalyzing one or more of a variety of reactions, including the ability to cleave, ligate or splice either themselves or other separate nucleic acid molecules in a nucleotide base sequence-specific manner. In general, enzymatic nucleic acids act by first binding to a target nucleic acid. Such binding occurs through a target-binding portion of the enzymatic nucleic acid molecule which is in close proximity to the enzymatic portion of the molecule that acts, for example, to cleave the target. Thus, the enzymatic nucleic acid first recognizes and then binds a target through complementary base-pairing, and once bound to the correct site, acts enzymatically upon the target. In one use, the enzymatic activity may involve a cleavage reaction. Strategic cleavage of a target RNA, for example, will destroy that RNA's ability to direct synthesis of an encoded protein. After an enzymatic nucleic acid has bound and cleaved its target, it is released from that molecule to search for another target and can repeatedly bind and cleave new targets. If the nucleic acid is cis-acting (e.g., self-cleaving, self-interacting, autolytic), then the activity is eliminated with the destruction of the nucleic acid. If the nucleic acid is trans-acting (cleaves or interacts with a another nucleic molecule or sequence), then the activity need not be eliminated with a single reaction.

The use of ribozymes has been proposed to treat diseases or genetic disorders by cleaving a target RNA, such as a viral RNA or a messenger RNA transcribed from a gene that should be turned off such as in cancer. These techniques have been described as an alternative to the blockage of the RNA transcript by the use of antisense sequences. Because of the enzymatic nature of certain ribozymes, a single ribozyme molecule may be used to cleave many molecules of target RNA, and therefore, therapeutic activity is achieved with relatively lower concentrations of material than required in an antisense treatment.

Because of their sequence specificity, trans-cleaving enzymatic nucleic acid molecules are studied and described as therapeutic agents to treat human disease. The enzymatic nucleic acid molecules can be designed or selected to cleave specific RNA targets within the background of cellular RNA. Such a cleavage event renders the mRNA nonfunctional and abrogates protein production from that RNA. In this manner, the synthesis of a protein associated with a disease state can be selectively inhibited. While there have been several descriptions of the use of ribozymes as therapeutic agents (Cotton, TIBTECH, 8:174-178, 1990; Usman & McSwiggen, Ann. Rep. Med. Chem. 30:285-294, 1995; Couture and Stinchcomb, TIG, 12(12):510-515, 1996; Christoffersen and Marr, J. Med. Chem. 38, 2023-2037, 1995; Gibson and Shillitoe, Molecular Biotechnology, 7:125-137, 1997; Persidis, Nature Biotechnology, 15:921-922, 1997; and Jaeger, Current Opinion in Structural Biology, 7:324-335, 1997) there have been relatively few studies of the use of trans- or cis-acting enzymatic nucleic acid molecules to alter gene expression (Chuat and Galibert, Biochemical and Biophysical Research Communications, 162(3):1025-1029, 1989; Innovir U.S. Pat. No. 5,741,679) or the control of gene expression by means of translation inhibition using small molecule RNA interactions (Werstuck and Green, Science, 282:296-298, 1998).

Researchers have demonstrated the remarkable diversity of catalytic functions which RNA molecules can perform besides the cleavage of other RNA molecules. See for example, Robertson and Joyce, Nature 344:467, 1990; Ellington and Szostak, Nature 346:818, 1990; Piccirilli, et al., Science 256:1420, 1992; Noller, et al., Science 256:1416, 1992; Ellington and Szostak, Nature 355:850, 1992; Bock, et al., Nature 355:564, 1992; Beaudry and Joyce, Science 257:635, 1992; and Oliphent, et al., Mol. Cell. Biol. 9:2944, 1989. RNA molecules with a given function, e.g., catalytic or ligand-binding, can be selected from a complex mixture of random molecules in what has been referred to as “in vitro genetics” (Szostak, TIBS 19:89, 1992) or “in vitro evolution”. In brief, a large pool of RNA molecules bearing random and defined sequences is synthesized and that complex mixture, for example, approximately 10¹⁵ individual sequences, is subjected to a selection and enrichment process. For example, by repeated cycles of affinity chromatography and polymerase chain reaction (PCR) amplification of the molecules bound to a ligand on the affinity column, Ellington and Szostak (1990) estimated that 1 in 10¹⁰ RNA molecules folded in such a way as to bind a given ligand. DNA molecules with such ligand-binding behavior have been isolated (Ellington and Szostak, 1992, supra; Bock et al., 1992, supra).

The control or regulation of gene expression is a highly desired objective in the fields of protein production, diagnostics, transgenics, cell therapy and gene therapy. A variety of expression control systems have been described as means to transcriptionally control the expression of a transgene in a recipient host cell. Control means or gene switches include, but are not limited to, the following systems.

Rapalogs may be used to dimerize chimeric proteins which contain a small molecule-binding domain and a domain capable of initiating a biological process, such as a DNA-binding protein or transcriptional activation protein (as described in WO 9641865 (PCT/US96/099486); WO 9731898 (PCT/US97/03137) and WO 9731899 (PCT/US95/03157)). The dimerization of the proteins can be used to initiate transcription of the transgene.

An alternative regulation technology uses a method of storing proteins, expressed from the gene of interest, inside the cell as an aggregate or cluster. The gene of interest is expressed as a fusion protein that includes a conditional aggregation domain which results in the retention of the aggregated protein in the endoplasmic reticulum. The stored proteins are stable and inactive inside the cell. The proteins can be released, however, by administering a drug (e.g., small molecule ligand) that removes the conditional aggregation domain and thereby specifically breaks apart the aggregates or clusters so that the proteins may be secreted from the cell. See Science 287:816-817 and 826-830, 2000.

Mifepristone (RU486) is used as a progesterone antagonist. The binding of a modified progesterone receptor ligand-binding domain to the progesterone antagonist activates transcription by forming a dimer of two transcription factors which then pass into the nucleus to bind DNA. The ligand binding domain is modified to eliminate the ability of the receptor to bind to the natural ligand. The modified steroid hormone receptor system is further described in U.S. Pat. No. 5,364,791; WO 9640911 and WO 9710337.

Yet another control system uses ecdysone (a fruit fly steroid hormone) which binds to and activates an ecdysone receptor (cytoplasmic receptor). The receptor then translocates to the nucleus to bind a specific DNA response element (promoter from ecdysone-responsive gene). The ecdysone receptor includes a transactivation domain/DNA-binding domain/ligand-binding domain to initiate transcription. The ecdysone system is further described in U.S. Pat. No. 5,514,578; WO 9738117; WO 9637609 and WO 9303162.

Another control means uses a positive tetracycline-controllable transactivator. This system involves a mutated tet repressor protein DNA-binding domain (mutated tet R-4 amino acid changes which resulted in a reverse tetracycline-regulated transactivator protein, i.e., it binds to a tet operator in the presence of tetracycline) linked to a polypeptide which activates transcription. Such systems are described in U.S. Pat. Nos. 5,464,758; 5,650,298 and 5,654,168.

Knockout and transgenic animals are well known to those skilled in the art. Swanson et al., Annu. Rep. Med. Chem., 29:265-274, 1994; Fassler et al., Int. Arch. Allergy Immunol., 106:323-334, 1995; Polites, H. G., Int. J. Exp. Pathol., 77(6):257-262, 1996; Harris and Foord, Pharmacogenomics, 1(4):433-443, 2000. A knockout animal has been genetically altered to disrupt the expression of a targeted gene, resulting in the elimination of the target gene product. Knockout animals are widely used to demonstrate the function of a protein of interest. In particular, the elimination of the expression of the targeted gene in the knockout animal can indicate the effect of inhibiting the protein product of the gene. One limitation with the technology is that targeted gene disruption can cause developmental defects which, although not indicative of the effect of target gene inhibition in an adult animal, result in embryonic lethality. Thus, certain disruptions of gene function cannot be studied in a viable animal. Another limitation of current knockout technology is the effect of developmental compensation on targeted gene disruption. In the course of development, other related gene products may compensate for the lost function of the disrupted gene and, thus, obscure its function in the adult animal. These well known limitations of the technology can significantly restrict the utility of targeted gene disruption in knockout animals. In the case of transgenic animals, an additional copy of the gene of interest is introduced into the organism and results in the over expression of the gene product. Protein over expression during development can cause defects or lead to compensation for or inhibition of over expression. These problems can obscure the effects of transgene over expression and limit the ability to interpret the biological effects of target gene over expression. The ability to create a conditional knockout animal is particularly important and relevant to overcome these limitations.

The references cited above are distinct from the presently claimed invention since they do not disclose and/or contemplate the identification of effectors and their use in the control of gene expression as provided by the instant invention. Nor do they involve allosteric control modules of the present invention.

### DESCRIPTION OF THE DRAWINGS

FIG. 1 is a diagrammatic representation of the present invention involving an allosteric control module containing a self-cleaving RNA domain, the activity of which is inhibited by interacting with an effector, thereby resulting in the translation of the mRNA. It will be appreciated by those skilled in the art that the allosteric control module may be placed 5′ or 3′ of the gene of interest.

FIG. 2 is a diagrammatic representation of the present invention involving an allosteric control module containing a self-cleaving RNA domain, the activity of which is initiated by interacting with an effector. The cleaving of the allosteric control module results in the degradation of the mRNA and inhibition of translation. In the absence of the effector, translation occurs.

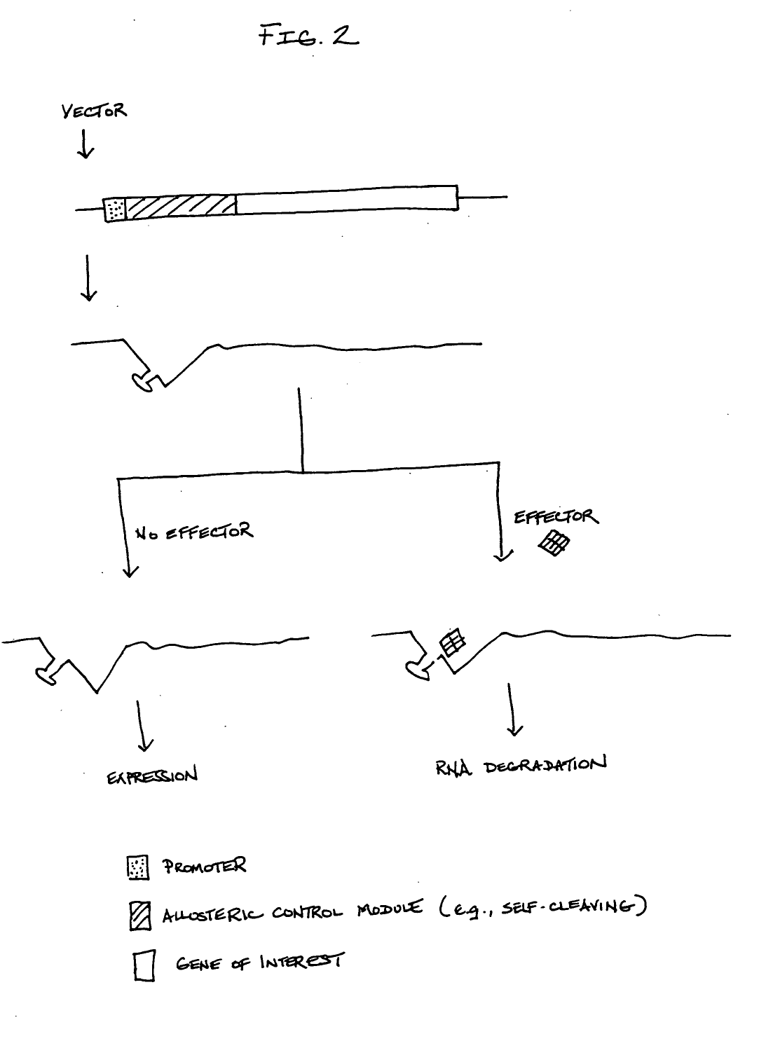

FIG. 3 is a diagrammatic representation involving an allosteric control module containing a self-splicing RNA domain, the activity of which is initiated by interacting with an effector. In this embodiment of the present invention, the presence of effector provides for the activation of the allosteric control module such that splicing of the precursor mRNA occurs to result in the formation of a translatable mRNA.

FIG. 4 is a diagrammatic representation involving an allosteric control module containing a self-splicing RNA domain in an engineered intron. In the depicted embodiment, the presence of effector provides for the activation of the allosteric control module such that splicing of the precursor mRNA occurs to result in the formation of a translatable open reading frame.

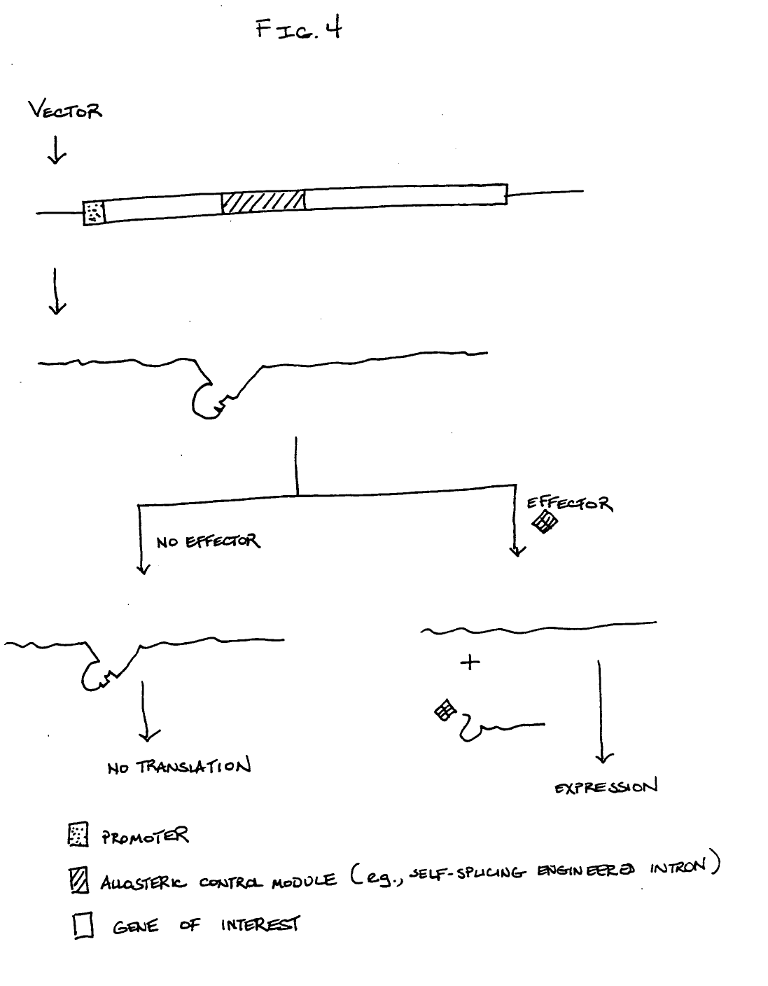

FIG. 5 is a diagrammatic representation of the present invention involving an allosteric control module containing a self-splicing RNA domain. In this embodiment, the activity of the allosteric control module is inhibited by interacting with an effector.

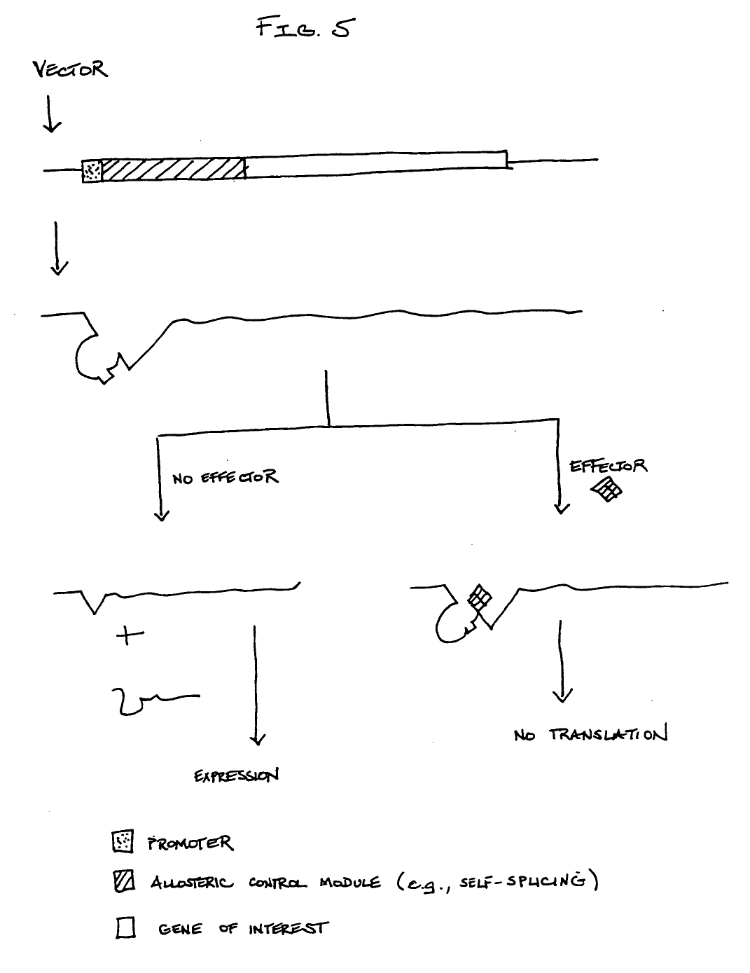

FIG. 6 is a diagrammatic representation of the present invention involving an allosteric control module containing a self-splicing RNA domain inserted in a region of an intron necessary for spliceosome assembly. In this embodiment, the activity of the allosteric control module is inhibited in the presence of effector, resulting in the expression of the gene of interest.

FIG. 7 depicts the secondary structure of RNA template 1 comprising a stem-loop structure of the theophylline-binding aptamer which is connected through a contiguous stretch of randomized nucleotides to a hammerhead ribozyme. The molecular structure of theophylline is also shown (SEQ ID NO: 6).

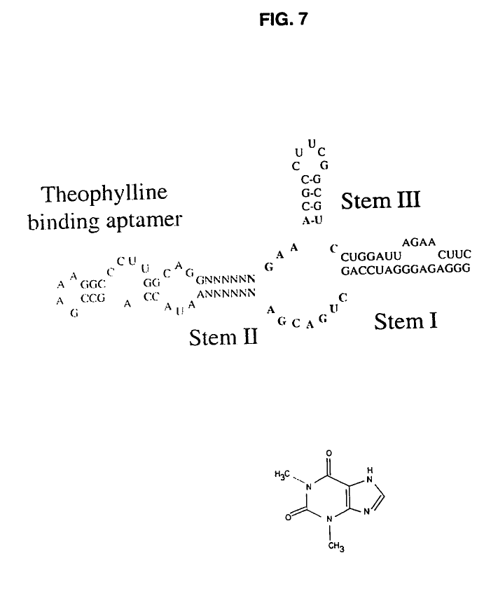

FIG. 8 depicts the secondary structure of the RNA sequence TA-50, selected after seven cycles.

FIG. 9 depicts the self-cleaving ability of TA-50 in the presence of theophylline.

### SUMMARY OF THE INVENTION

The means to identify a useful effector molecule is described as is the use of the identified effector to evolve a cognate aptamer. The construction of an allosteric control module is described in which a catalytic RNA forms a part of or is linked to the effector-binding RNA domain or aptamer, thereby placing the activity of the catalytic RNA under the control of the effector and requiring the presence of the effector for activation or inactivation. RNA molecules are constructed in which at least one portion is capable of binding an effector and another portion is a catalytic RNA. The present invention involves both the evolution of RNA sequences which bind the effector and a selection process in which the allosteric control modules are identified by their catalytic function in the presence and absence of the effector. In this manner, regulatable catalytic RNAs may be selected for use in cleaving, splicing or ligating a target RNA in the presence of an effector, or in cleaving, splicing or ligating a target RNA in the absence of an effector.

These methods of effector selection and the construction of allosteric control modules are useful in altering the expression of a target RNA molecule in a controlled fashion. It is particularly useful when the target RNA molecule is formed in or delivered to the cell in combination with the allosteric control module.

### DETAILED DESCRIPTION OF THE INVENTION

Disadvantages associated with previously known control constructs and their uses include potential toxicities in in vivo use, including the transcriptional activation or repression of endogenous genes, the activation or inhibition of proteins or cellular processes that the small molecule entity normally regulates, or the induction of an immune response towards the foreign proteinaceous gene products of the control system. In addition, there may be size constraints upon the control coding sequences that can be delivered together with the gene of interest by means of certain viral and non-viral vectors. Thus, there is still a need to develop alternative methods and materials for the controlled expression of a gene of interest. Furthermore, the development of a means of controlling gene expression wherein that means may also be varied by the individual components used would contribute significantly to any strategy of gene therapy as well as to the production of therapeutic proteins. The present invention uniquely solves these problems.

The expression of a specific gene can be altered at any step in the process of producing an active protein. The modulation of total protein activity may occur via transcriptional, transcript-processing, translational or post-translational mechanisms. Transcription may be modulated by altering the rate of transcriptional initiation or the progression of RNA polymerase. Transcript-processing may be influenced by circumstances such as the pattern of RNA splicing, the rate of mRNA transport to the cytoplasm, or mRNA stability. The present invention primarily concerns the identification and use of effector molecules and the creation of allosteric control module molecules which act together to alter the expression of a target gene (for example, altering the in vivo concentration of a target protein by altering RNA processing.) The present invention provides an identification process for suitable effectors for use in the control of gene expression, which process has not been previously described.

Descriptions of various effector-controlled RNA molecules are available. See for example, Chuat and Galibert, Biochemical and Biophysical Research Communications, 162(3):1025-1029, 1989; Ellington et al., Nature 355:850-852, 1992; Porta et al., Bio/Technology 13:161-164, 1995; and Soukup and Breaker, Proc. Natl. Acad. Sci. USA, 96:3584-3589, 1999. The identification and in vitro selection processes, however, have been designed for and directed to the use of ribozymes to control the expression of deleterious genes (such as HIV, HCV, CMV, VEGF and TNF) or for biosensors to detect specific ligands. Aminoglycoside antibiotics are among the most studied molecules which react with RNAs (von Ahsen et al., Nature 353:368-370, 1991; von Ahsen et al., J. Mol. Biol. 226:935-941, 1992; Murray and Arnold, Biochem. J. 317:855-860, 1996; Werstuck and Green, Science 282:296-298, 1998). Such previously described molecules, however, react directly with naturally occurring ribozymes. The present invention describes the novel identification of effectors which are used to evolve aptamers for use in expression regulation constructs.

Alternatively, Werstuck and Green (Science, 282:296-298, 1998) described the use of effectors and aptamers to regulate translation. In that research they described the use of multiple aptamers and dyes as effectors to repress the translation of a reporter gene. The method did not involve the use of a catalytic RNA or allosteric control module of the present invention. While other disclosures relate to the use of regulated gene expression using a ligand, ligand binding sequence and catalytic RNA (Innovir Laboratories Inc.; expression control systems and nucleic acid constructs are described in U.S. Pat. No. 5,741,679 and U.S. Pat. No. 5,834,186), the descriptions have not included a process for the selection of suitable effectors.

The present inventors are not aware of any prior reports of a systematic process for the identification of effectors, their use to select and evolve non-naturally occurring aptamers for the construction of allosteric control modules, and the combined use of effector and allosteric control module for the alteration of gene expression. Such a process is an object of the present invention. The procedures described herein also serve to produce molecules not previously envisioned in the regulation of gene expression. The present invention provides a means for determining whether a “non-therapeutic” molecule can specifically modulate the expression of a gene of interest, and the ultimate clinical use of such an effector molecule provides an advantage over the use of previous biologicals, or drugs which have a therapeutic function.

Methods and compositions are described for the controlled expression of targeted RNA molecules by means of an allosteric control module. The activity of the allosteric control module is altered through the presence, absence or amount of a pre-identified effector. In one embodiment, the allosteric control module is active in the presence of an effector, in another embodiment the allosteric control module is inactive in the presence of an effector.

The following definitions are used in the description of the present invention.

An “allosteric control module” as used herein refers to a non-naturally occurring RNA composed of at least two domains, one a receptor for a pre-identified ligand and the other a catalytic domain. The receptor domain or effector-binding domain may also be referred to as an aptamer, and the ligand to which it binds is referred to as an effector. The catalytic domain is a RNA which is capable of interacting with a target RNA. Such activity may include cleaving, splicing, or ligating the target RNA.

The allosteric regulation of gene expression refers to the alteration of the expression of a gene, preferably a transgene in gene therapy, by means of the interaction of the effector and allosteric control module. The effectors of the present invention bind to a domain of the allosteric control module and alter the activity of the module's catalytic domain. Without being bound by any particular theory, it is believed that the influence of the effector upon the activity of the catalytic domain is brought about by a change in the conformation of the module which change is induced by the binding of the effector and the effector-binding domain or aptamer. The combination of the aptamer and catalytic RNA domain is selected such that the conformational change may either preclude formation of an active catalytic domain or induce the formation of an active catalytic domain. For example, the effector-induced conformational change may be selected to cause the inhibition of or a reduction in the activity of the catalytic domain due to steric interference between the aptamer and the catalytic domain tertiary structures. Alternatively, the effector-induced conformational change may be selected to cause the initiation of or an increase in the activity of the catalytic domain. Therefore, the term “activation” or “activated” is used herein to refer to either the initiation of or an increase in or enhancement of catalytic activity.

It will be appreciated by those skilled in the art that the domains of the allosteric control module may be non-overlapping or partially overlapping such that one or more domains are encoded in part by the same polynucleotide. Thus, the domains are primarily distinguished by their function rather than by their sequence. In addition, the domains may be separately prepared and then joined to form the allosteric control module, or the allosteric control module may be prepared as a single polynucleotide having both aptamer and catalytic domains.

In contrast to previously described ribozymes and ribozyme-like molecules, the allosteric control module of the present invention does not have true “enzymatic” activity. In a preferred construction of the DNA of the present invention, the allosteric control module acts on an intramolecular basis and is only required to provide one reaction rather than multiple reactions with multiple RNA or DNA molecules. In addition, the allosteric regulation of the catalytic function differs from that of inhibitors which block the catalytic sites of structures such as a ribozyme. In the present invention, the effector binds to an aptamer which is a site located apart from the active site, and its influence on the activity of the polynucleotide is thought to be brought about by changes in polynucleotide conformation which result from the interaction of the aptamer and effector. It will also be appreciated that the position of the allosteric control module in the DNA constructs of the present invention may vary. All that is required is that the allosteric control module be positioned such that altering the activity of the allosteric control module by means of an effector will result in the alteration of expression of the gene of interest.

“Allosteric” or “allostery” as used herein refers to the alteration of the activity of a molecule by the interaction of an effector with that molecule. The effector interacts a domain distinct from the molecule's catalytic domain and that interaction causes a change in the activity of the molecule. The typical activity involved in the constructs of the present invention may be referred to as catalytic activity, which is further described herein.

An “aptaner” or “effector-binding domain” as used herein refers to a polynucleotide that binds to an effector. The polynucleotide typically comprises at least 20 nucleotides and may comprise at least 300 nucleotides. The aptamers of the present invention may comprise naturally occurring polynucleotides, which are not chemically modified. Preferably, the aptamer comprises a synthetic or non-naturally occurring polynucleotide. In addition, the aptamer is preferably a ribonucleic acid (RNA) selected by in vitro evolution to interact with a previously identified effector. The in vitro evolution of aptamers begins with a pool of RNA molecules created by chemical and/or enzymatic synthesis. Typically, the desired aptamer is selected based upon its ability to interact (e.g., recognize and bind) with an effector. Thus, in a preferred embodiment, the effector is a predetermined molecule which is used to select and further evolve a suitable aptamer. Alternatively, the aptamer may be constructed and libraries of molecules screened to identify and select a suitable effector.

For use in the control of gene expression in gene therapy techniques, the aptamer is not an isolated and purified chemical entity. Instead, the aptamer is encoded by a DNA which is delivered to a cell, and the aptamer becomes a portion of the mRNA transcribed from that DNA in the host cell. In addition, the effectors of the present invention do not modify a biological activity of an aptamer because the aptamers of the present invention have no inherent physiological activity in the recipient cell.

There is no set number of bases required for the interaction (e.g., hybridization or binding) of an aptamer to an effector. In general the aptamer will contain 20 to 300 nucleotides, selected as described herein for binding to a specific effector. The small size of the molecule (typically 200 nucleotides or less, preferably between 20-30 nucleotides in length) is advantageous for cell delivery as compared with conventional expression regulation constructs which are much larger in size. Although referred to herein as a “random” sequence, it is understood that the RNA is random only as originally used in the evolution process. The product of the evolution process, i.e., the aptamer(s), is not a random sequence. It is a specific sequence which binds with a high degree of affinity and specificity to a defined effector.

The specific aptamers of the allosteric control modules described herein are not limiting in the invention. Those skilled in the art will recognize that all that is important in an aptamer of the present invention is that it selectively and specifically interact with a suitable effector, and that it have the ability to alter the catalytic activity of the allosteric control module when the aptamer has interacted with the effector. Multiple aptamers (as well as multiple allosteric control modules) may also be used so that multiple effectors and even multiple different effectors may be used to react with allosteric control modules and thereby alter gene expression by altering the precursor mRNA.

The term “domain” as used herein refers to a polynucleotide which provides a selected activity or function to the allosteric control modules of the present invention.

An “effector” as used herein refers to a molecule which interacts with an aptamer. Binding may be the result of the interaction and may include, but is not limited to, hydrogen binding, hydrophobic interactions, intercollations, etc. Suitable molecules for use as effectors of the present invention include, but are not limited to, organic or inorganic molecules, peptides, polypeptides, proteins, oligonucleotides, polynucleotides, nucleic acids, naturally occurring metabolites and biological effectors, lipids, carbohydrates (polysaccharides, sugar), fatty acids, and polymers. The preferred effector molecules of the present invention are distinguished from those described in the art in that the present effectors have either no pharmalogical effect or are used at concentrations wherein a pharmalogical effect either is not observed or is negligible.

“Suitability” of the effector for use with the allosteric control module may include the following characteristics. (1) The effector has little or no pharmalogical effect in the dosage range used in altering gene expression or has a negligible pharmalogical effect. This refers to either the lack of any alteration in function of the structure or process of a cell in which the effector acts (other than the allosteric control module) or the occurrence of a insignificant alteration in function of the structure or process of a cell in which the effector acts (other than the allosteric control module). In other words, if the effector may be used as a pharmaceutical agent that has an effect on a structure or process other than the allosteric control module, then that effect does not cause any harm to the patient not in need of treatment by that pharmaceutical agent. (2) The effector will undergo biodistribution to that cell or tissue which will contain the allosteric control module. (3) The effector has the ability to pass to subcellular structures, i.e., to the allosteric control module in the nucleus of cells transformed for regulated gene expression. This may be by means of intracellular diffusion or transport, and most preferably, by intranuclear diffusion or transport. (4) The effector has the ability to interact with an aptamer wherein the interaction occurs with high specificity and a high affinity.

Additional considerations for the identification of a suitable effector may further include the following. (1) If the effector is a molecule which has a dose-effect relationship, then the molecule typically is used at a daily maximum dose which is less than the usual daily minimum dose of the molecule when used for an approved indication. Preferably such an effector is used at a dosage level which is below 25% of the effective dose (ED25) of the molecule when used as a pharmaceutical agent. It will be appreciated by those skilled in the art, however, that such a preference is based on a case-by-case evaluation of the agent. For example, in the case of an effector which is an antiviral agent, the effector could be used at any dosing range in the absence of the virus. More preferably the dosage level is below the ED10 for the molecule. Most preferably, if the effector is a molecule which has a dose-effect relationship, then the molecule is used as an effector at a dose which is below the lowest effective dose (threshold dose) of the molecule when used as a pharmaceutical agent. (2) The use of a pharmaceutical agent related to the effector is not contraindicated in patients having the condition to be treated by the regulated gene. (3) There are no significant side effects or adverse reactions produced by the effector itself, and no significant adverse reaction is due to an overdose of the effector itself. Typically, non-significant effects would include, for example, events such as headache, dizziness, lightheadedness, sedation, nausea, vomiting, rash, constipation, diarrhea, abdominal pain, euphoria, dysphoria, fatigue, arthralgia which can be controlled by dose adjustment or other common intervention or which occurs in less than five percent of the population receiving the effector. (4) There are no known contraindications in pregnancy, heart disease, or hypersensitivity to related agents.

In certain preferred embodiments of the present invention, the effector is a small molecule. The nature of the effector can be chosen to be exogenously supplied, such as some non-toxic molecule or drug which readily enters at least the cells containing the targeted RNA. Alternatively, an entirely endogenous system can be used in which the controlling effector is some endogenous metabolite or macromolecule which is directly or indirectly related to the pathology to be corrected or the gene to be expressed or the molecule to be produced. For example, a protein encoded by the target RNA could be the effector. The construct may be designed such that the activity of the allosteric control module is dependent on binding to the expressed protein and as the level of protein increases the activity of the module increases to cause a decrease in expression. As the level of target RNA falls due to alteration (e.g., cleavage) by the allosteric control module, the concentration of the protein (as ligand or effector) also falls. When the concentration falls below that at which the regulatable RNA molecules are all occupied, the rate of alteration will begin to fall off. By selecting for differing aptamer-effector affinities, the appropriate level of regulation of allosteric-mediated destruction of the target RNA can be achieved for any given situation.

The “pharmalogical activity” of a molecule is used to refer to the activity of that molecule as a drug or medication.

A “database” as used herein refers to any compilation of information on potential effectors, such as small molecules, containing information concerning the suitability of the effector for use in controlling gene expression.

The “catalytic activity” of the allosteric control module refers to the activity of the “catalytic domain” or “catalytic sequence” which is a nucleic acid which acts on a target nucleic acid in a desirable manner. Examples of possible actions include, but are not limited to binding of the target, reacting with the target in a way which modifies/alters the target as by cleavage, splicing or ligation or the functional activity of the target, or facilitating the reaction between the target and another molecule. In preferred embodiments of the present invention, the catalytic activity is a self-cleaving activity, a ligase activity, or a splicing activity. Such activities are often associated with ribozymes. Ribozymes, including ribozyme-like molecules and portions of such molecules, may be used to form the catalytic domain of the allosteric control module of the present invention. It will be appreciated by those skilled in the art that it is primarily the catalytically active portion of the naturally occurring ribozyme or non-naturally occurring ribozyme-like molecules that is used in the allosteric control modules of the present invention, but that additional domains also may be used. For example, if the allosteric control module is self-cleaving, then in addition to an aptamer and a catalytic domain, the module will further include a substrate domain. It will also be appreciated that for the purposes of the present invention, the “catalytic activity” of the allosteric control module merely refers to the alteration or modification of an interaction with a target RNA. The catalytic domain may be designed such that it may or may not be consumed in the process, and therefore, the domain is not required to be a true “catalyst”.

Ribozymes which may be useful to the present invention in the preparation of catalytic domains include, but are not limited to, molecules in the classes of hammerhead, axehead, hairpin, hepatitis delta virus, neurospora, self-splicing introns (group I or group II), ligases, phosphatases and polymerases. Each class of ribozyme cleaves a different sequence of nucleotides using distinct mechanisms of action. Moreover, each class is further distinguished based on how many nucleotide bases are essential for catalytic activity and to the extent the intended target sequence and the ribozyme can be manipulated to alter specificity. Thus, naturally occurring ribozymes may be used as a basis to create non-naturally occurring (totally unique or modified) ribozyme-like structures. For simplicity, the term ribozyme may be used in place of catalytic domain in the present invention, but it will be appreciated that the catalytic domain need not contain all of a naturally occurring ribozyme structure to be used in the present invention, and it may involve a synthetic ribozyme-like structure.

The term “RNA ligase” or “ligase domain” as used herein refers to a polynucleotide capable of catalyzing (altering the occurrence, velocity and/or rate of) a ligation reaction (the joining together of two polynucleotides) in a nucleotide base sequence-specific manner. As with other catalytic domains, the ligase domain may act in cis, i.e., as an intramolecular reaction, or in trans, i.e., as an intermolecular reaction when the gene of interest, the expression of which is to be regulated, is provided to the cell as a separate DNA construct. The RNA ligase may have complementarity in a substrate binding region to a specified target polynucleotide, and also has a catalytic activity to specifically join RNA in that target. By “complementarity” is meant a nucleic acid that can base pair (e.g., form hydrogen bond(s)) with other RNA by either traditional Watson-Crick or other non-traditional types (for example, Hoogsteen type) of base-paired interactions. This complementarity functions to provide sufficient hybridization of the ligase domain to the target RNA to allow the reaction to occur. Complementarity of 100% is preferred, but complementarity as low as 50-75% may also be useful. The nucleic acids may be modified at the base, sugar, and/or phosphate groups. The specific ligase domains of the allosteric RNA ligase polynucleotides are not limiting in the invention, and those skilled in the art will recognize that all that is important in a ligase domain of this invention is that it has a specific substrate binding site which is complementary to one or more of the target polynucleotide regions, and that it include a site within or surrounding that substrate binding site which imparts a ligase activity to the ligase domain. It will be appreciated by those skilled in the art that catalytic domains having cleaving or splicing activities may also involve complementarity in a substrate binding domain to a specified target polynucleotide in order to specifically interact the catalytic RNA and the desired target.

The term “gene of interest” or “desired gene” as used herein refers to a gene providing a physiologically relevant benefit to the cell or organism, such as encoding a therapeutically relevant molecule for which expression is controlled by means of the effector and allosteric control module. For example, a therapeutic gene of interest is a gene that corrects or compensates for an underlying protein deficit or, alternately, that is capable of down-regulating a particular gene. Moreover, a gene of interest can be a gene that mediates cell killing, for instance, in gene therapy for the treatment of cancer. The term “transgene” as used herein refers to a gene, such as the gene of interest, which is transferred to a cell.

The term “messenger RNA” (mRNA) is used to refer to a polynucleotide which transfers information from DNA to the protein-forming system of the cell. The term “precursor mRNA” or “pre-mRNA” is used to refer to a polynucleotide which is directly transcribed from the coding DNA strand, may contain an intron or introns, and may or may not be capped with an inverted methylated guanosine nucleotide.

The term “non-natural” polynucleotide as used herein refers to a polynucleotide sequence or construct that does not occur in nature. The preferred allosteric polynucleotides of the present invention do not occur in nature. The term “isolated polynucleotide” refers to a nucleic acid molecule of the invention that is free from at least one contaminating nucleic acid molecule with which it is naturally associated, and preferably substantially free from any other contaminating mammalian nucleic acid molecules which would interfere with its use in protein production or its therapeutic or diagnostic use.

The term “splice recognition region” as used herein refers to a sequence in the precursor RNA which serves as a splice donor, splice acceptor or spliceosome binding site. The splice donor is typically a site at the 3′ end of the exon (located at the 5′ end of the intron to be removed), and the splice acceptor refers to a site at the 5′ end of the adjacent exon to be ligated (the 3′ end of the intron to be removed). “Spliceosome” refers to a large, multicomponent complex of cellular protein and RNA that binds to and directs the processing of the pre-RNA into mRNA by cleavage, the removal of introns, and the ligation of exons.

The term “intron” is used to refer to a section of RNA occurring in a transcribed portion of a gene which is included in a precursor mRNA but which is then excised during processing of the transcribed RNA before translation occurs. Therefore, intron sequences are not found in mRNA nor translated into protein.

The term “exon” is used to refer to a portion of a gene that is represented in the transcript of the gene and that survives processing of the RNA in the cell to become part of a mRNA. Exons generally encode three distinct functional regions of the RNA transcript. The first region, located at the 5′ end which is not translated into protein, is called the 5′-untranslated region (5′-UTR). The 5′-UTR signals the beginning of RNA transcription and contains sequences that direct the mRNA to the ribosomes and cause the mRNA to be bound by ribosomes so that protein synthesis can occur. The second region is known as an open reading frame and contains the information that can be translated into the amino acid sequence of the protein or function as a bioactive RNA. The third region, located at the 3′ end, contains the signals for the termination of translation and for the addition of a polyadenylation tail (poly(A)) and is not translated into protein (i.e., the 3′-UTR). In particular, the 3′-UTR can provide mRNA stability. The intron/exon boundary will be that portion in a particular gene where an intron section connects to an exon section. The terms “TATA box” and “CAP site” are used as they are recognized in the art.

The term “vector” is used to refer to any molecule (e.g., nucleic acid, plasmid, virus, small molecule, liposome, carrier molecule, etc.) used to transfer coding information to a host cell.

The terms “control sequences” and “control elements” are used to refer collectively to non-coding regulatory sequences including, but not limited to, promoters, polyadenylation signals, transcription termination sequences, upstream regulatory domains, origins of replication, internal ribosome entry sites, enhancers, and the like, which are operably linked to the DNA encoding a gene of interest to provide for the transcription and translation of the coding sequence in a recipient cell. Not all of these control elements need always be present so long as the sequence encoding the gene of interest is capable of being transcribed and translated in an appropriate host cell in accordance with the expression regulatory means of the present invention.

A “promoter” is used to refer to a DNA sequence that directs the binding of RNA polymerase and thereby promotes RNA synthesis. An “origin of replication” is a sequence on a vector or host cell chromosome that renders extragenomic elements (e.g., viruses or plasmids) capable of replicating independently of the host cell genome. “Enhancers” are cis-acting elements of DNA that stimulate or inhibit transcription of adjacent genes. An enhancer that inhibits transcription is also termed a “silencer”. Enhancers differ from DNA-binding sites for sequence-specific DNA binding proteins found only in the promoter (which are also termed “promoter elements”) in that enhancers can function in either orientation, and over distances of up to several kilobase pairs, even from a position downstream of a transcribed region.

The term “operably linked” is used to refer to an arrangement of elements wherein the elements so described are configured or assembled so as to perform their usual function. Thus, a control sequence operably linked to a coding sequence is capable of effecting the replication, transcription and/or translation of the coding sequence. For example, a coding sequence is operably linked to a promoter when the promoter is capable of directing transcription of that coding sequence. The control sequence need not be contiguous with the coding sequence, so long as it functions correctly. Thus, for example, intervening untranslated yet transcribed sequences can be present between a promoter sequence and the coding sequence and the promoter sequence can still be considered “operably linked” to the coding sequence.

The term “co-element” as used herein refers to a separate molecule or a separate domain in the precursor mRNA which interacts with or complexes with the catalytic domains and/or aptamers of the allosteric control module and/or the effector to yield a catalytic complex. For example, the co-element may complete a missing portion of the allosteric control module so that it becomes catalytically active. Such co-elements include components as described in WO 9808974. Alternative co-elements may include the bridge elements described by Soukup and Breaker (Proc. Natl. Acad. Sci. USA 96:3584-3589, 1999) to construct ligand responsive allosteric ribozymes.

The term “gene transfer” or “gene delivery” is used to refer to methods and/or systems for reliably inserting a particular polynucleotide (e.g., DNA) into a target cell. Gene transfer may take place in vivo (e.g., adeno-associated virus gene therapy) or ex vivo (e.g., as with extracellular modification of cells with a retrovirus followed by transfer or implantation of the transformed cells into the host). Such methods may result in the integration of the transferred genetic material into the genome of target cells or the transferred genetic material may function independently of the host cell genome.

The terms “reduction”, “inactivation”, “inhibition”, “initiation” and “decrease” in expression as used herein means that the level of translation of a target mRNA is reduced below that observed in the absence of the regulation means of the present invention. Thus, the inhibition of gene expression may range from a decrease in the translation of the target mRNA to the complete inactivation or inability of the transcript to be used to express the gene of interest.

The terms “enhancement”, “activation”, “induction” or “increase” in expression as used herein means that the level of translation of a target mRNA is increased above that observed in the absence of the regulation means of the present invention. Thus, induction of expression may range from the initiation of the translation of the target mRNA to an increase in the translation of the target mRNA.

The phrase “specifically controlling the expression of the gene of interest” as used herein means altering the expression of the gene of interest without altering the expression of other genes in the cell in a way which would cause an adverse effect on (a) an organism containing the cell in the case where the cell is within the organism or (b) the growth or the culturing of the cell, in the case where the cell is being grown or cultured to make a product where the amount of product produced is associated with expression of the gene of interest.

Alteration of Gene Expression

The production of a recombinant protein or peptide can be affected by the efficiency with which DNA (or an episomal nucleic acid) is transcribed into mRNA. Conventional control systems seek to affect the transcription event. The production of a recombinant protein or peptide, however, can also be affected by the efficiency with which precursor mRNA is modified to form the mRNA which is translated into protein. The novel constructs and methods of the present invention advantageously provide for the regulated expression of a gene of interest, such as a therapeutic protein, by altering the process of pre-mRNA processing.

The allosteric control modules of the present invention contain catalytic and effector-binding domains that are specifically selected such that the interaction of the effector-binding domain or aptamer with an effector alters the activity of the allosteric control modules. For example, the interaction of the effector and aptamer may result in a conformational change in the allosteric control module. Depending upon the selection of the allosteric control modules, the conformational change can result in either an increase or a decrease in the activity of the catalytic domain of the module. This in turn affects whether or not the precursor mRNA is appropriately modified to form a mRNA capable of being translated into a protein of interest. While not limiting to the present invention, a conformational change may be caused by the binding energy derived from the effector-aptamer interaction which is used to shift the thermodynamic balance between two possible confirmations of the allosteric control module. Depending upon the format design, as described in greater detail in the different embodiments, the allosteric control module is either activated or inactivated by the effector.

The present invention includes an expression regulation format involving an allosterically activated self-cleaving RNA as the catalytic domain of the allosteric control module. In one aspect of this embodiment, the allosteric control module may be encoded in a mRNA with the gene of interest. For example, the DNA sequence is designed to encode a self-cleaving site that separates the cap and message sequences. As described in further detail herein, the aptamer and catalytic domain may be selected such that in the absence of an effector, the catalytic domain is active and that activity results in the cleavage of the pre-mRNA or mRNA. Thus, the gene of interest is not expressed because the mRNA can not be translated. In the presence of the effector, the catalytic domain is inhibited and thus inactive or unable to act upon the RNA substrate. As a result, the mRNA is not cleaved. Thus, the gene of interest is expressed because the RNA can be appropriately processed and translated. A preferred embodiment of the present invention involves constructs which provide gene expression in the presence of the effector.

In an alternative embodiment involving an allosterically activated self-cleaving RNA, the aptamer and catalytic domain may be selected such that in the presence of an effector, the catalytic domain is active and that activity results in the cleavage of the mRNA. Thus, the gene of interest is not expressed because the mRNA can not be translated. In the absence of the effector, the catalytic domain is inactive or unable to act upon the RNA substrate. As a result, the mRNA is not cleaved and the gene of interest is expressed. Thus, in the absence of effector the gene is expressed.

In yet another embodiment the catalytic domain may include an engineered intron which contains a self-cleaving site. The aptamer and effector are selected such that the domain does not cleave if effector is absent. As a result, the mRNA is not recognized as an active molecule and is not translated.

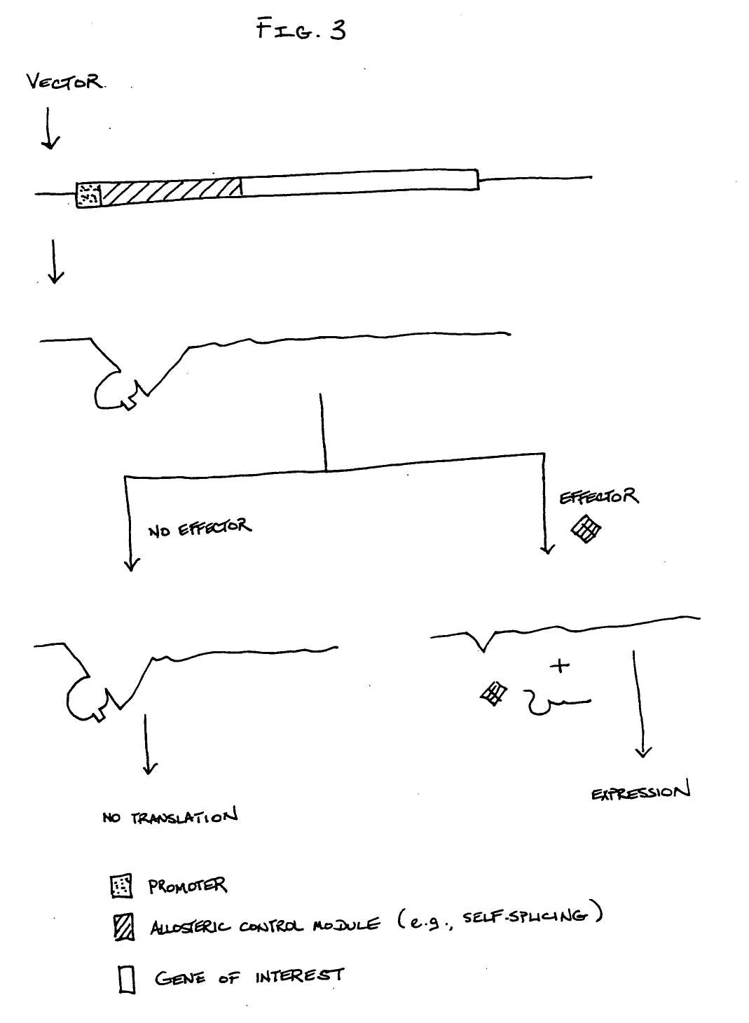

The present invention also includes an expression regulation format involving an allosterically activated splicing nucleic acid as the catalytic domain having cleaving and ligase activities. In one aspect of this embodiment, the allosteric control module is inserted into an exon of the gene of interest. This results in the formation of a “engineered intron” (eI) as depicted in FIG. 3. In the absence of the effector, the allosteric control module is inactive, thus leaving the engineered intron in place and resulting in an altered open reading frame that leads to the inhibition or reduction of protein expression. In the presence of the effector, the allosteric control module is active. This activity results in the removal of the engineered intron and the ligation of the exon to form a productive open reading frame that leads to the activation or increase in protein expression.

The presence of the eI in the pre-mRNA can result in the inhibition of gene expression by a variety of mechanisms that can act individually or jointly. Such mechanisms include, but are not limited to 1) the eI may be designed to contain several stop codons which would arrest translation; 2) the eI could code for nonsense amino acids resulting in a protein with multiple inactivating mutations; and 3) the presence of the eI could activate the mRNA surveillance system native to cells that would sequester or destroy the altered pre-mRNA. It will be further appreciated that the format may involve an engineered intron designed such that in the presence of effector the allosteric control module is inactive and in the absence of the effector the module is active.

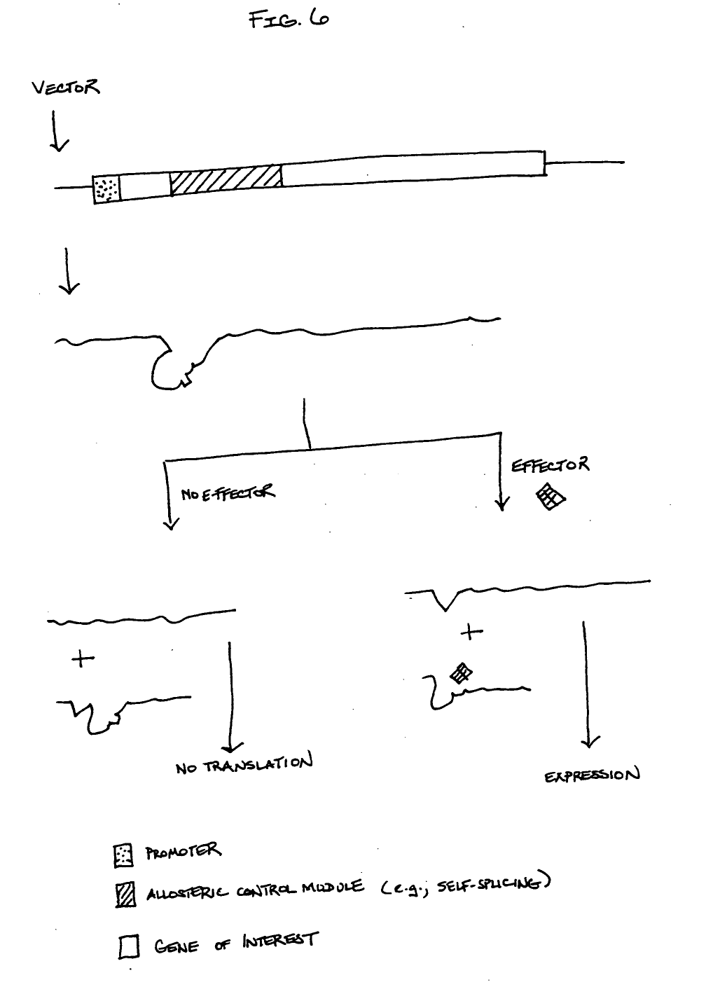

The present invention further includes an expression regulation format involving an inhibitable allosteric self-splicing intron. In this embodiment, the allosteric control module is inserted into an intron in a region necessary for spliceosome assembly such that the action of the self-splicing intron results in the removal of a nucleotide sequence necessary for normal splicing. This embodiment is depicted in FIG. 6. In the absence of effector, the allosteric control module is active, thus leading to the removal of the vital splice recognition site. The removal of the splice recognition site results in an altered open reading frame that leads to the inhibition or reduction of protein expression. In the presence of effector, the allosteric control module is inactive and, as a result, normal splicing occurs at the splice recognition sites, the correct open reading frame is formed, and the protein is expressed.

Effector Selection

A fundamental aspect of the present invention is the identification of an effector for use with an allosteric control module to alter the expression of a gene. Associated with such a use is the use of the effector to evolve a cognate aptamer which will form a part of the allosteric control module. The present invention provides a novel, systematic method for identifying an effector and generating an interactive aptamer (or aptamers). The method involves the following steps. First, the desired characteristics for the effector are selected. These desired characteristics are selected from one or more of the following attributes which are useful in identifying a potential effector suitable for use in protein expression and gene therapy techniques:
a) has at least 1% bioavailability;
b) is biodistributed to tissue containing an allosteric control module;
c) has the ability to pass to the nucleus of the cell;
d) exhibits either no drug interactions or manageable drug interactions;
e) exhibits either no toxicity or acceptable toxicity at the dosage range used;
f) exhibits either no side effects or acceptable side effects at the dosage range used;
g) exhibits either no pharmacological effect at the dosage range used in regulating transgene expression or a negligible pharmacological effect; and
h) possess physical properties suitable for the in vitro evolution of an aptamer (desirable physical properties may include a planar molecule which has a rigid structure.)

These characteristics indicate that the effector is suitable for aptamer generation, human consumption and use with an allosteric control module for the regulation of gene expression. Information on these characteristics may be obtained by accessing one or more databases containing data on the selected characteristics. Databases containing the relevant information include, but are not limited to, Investigational Drugs database (IDdb, Current Drugs; Current Drugs Ltd., Philadelphia, Pa.), Drug Data Report (MDDR, MDL Information Systems Inc., San Leandro, Calif.; Prous Science Publishers, Barcelona, Spain), World Drug Index (WDI, Derwent Information, Alexandria, Va.) and Derwent Drug File, R&D Insight (Adis International Inc., Langhorne, Pa.), R&D Focus (IMS HEALTH, IMSworld Publications Ltd., London, England), Pharmaprojects (PJB Publications, Surrey, United Kingdom), MEDLINE (The National Library of Medicine) and EMBASE (Elsevier Science, B.V). A set of effectors having the selected characteristics is then identified.

“Bioavailability” as used herein refers to the ability of the effector to reach its intended site of action after administration. The effector will have a bioavailability of at least 1%. Preferably, the effector will have a bioavailability of at least 5%, and more preferably the effector will have a bioavailability of at least 10%. A preferred effector will also be bioavailable upon oral delivery, but it will be appreciated that the route of delivery is not limiting to the present invention. The effector may be administered parenterally, e.g., by injection intravenously, intraperitoneally, intramuscularly, intrathecally or subcutaneously. The selected effector may be formulated as a composition for oral administration (including sublingual and buccal), pulmonary administration (intranasal and inhalation), topical administration, transdermal administration, and rectal administration. Delivery may involve a single dose schedule or a multiple dose schedule.

Using this method, suitable effectors may be identified from a variety of molecules including, but not limited to, small organic molecules, peptides, polypeptides, proteins, oligonucleotides, polynucleotides, nucleic acids, naturally occurring metabolites and biological effectors, lipids, carbohydrates (polysaccharides, sugar), fatty acids, and polymers. In a preferred embodiment of the present invention, the effector is a small molecule.

It will be appreciated that any database, or combination of databases, may be used in the performance of the present invention. Suitable databases of potential effectors will include molecules such as:
a) marketed drugs with stereoselectivity for an isomer that comprises the pharmaceutically active component and another isomer with little or no pharmacological activity (the latter being the possible effector of interest);
b) known drug metabolites having little or no activity;
c) nuclear receptor targeted molecules (for example, Vitamin D, retinoic acid, steroids);
d) drug candidates which entered clinical trials, but the trials were discontinued due to a relative lack of efficacy;
e) drugs that were removed from the market because of lack of therapeutic efficacy;
f) drugs that are efficacious but which are not marketed because of low relative benefit;
g) drugs designed as antiviral/anti-infectives, for use in patients not affected by the targeted virus or infectious agent;
h) well characterized food additives;
i) generic drugs with well known mechanisms of action; and
j) drugs that were displaced from the market or clinical trials by best in class molecules.

The identified effectors may then be used to generate and select aptamers to the effectors in the set by means of in vitro evolution. The combinations of effectors and aptamers, as well as effectors and allosteric control modules, may then be evaluated to identify those molecules best suited for the control of gene expression.

Beyond allosteric control module binding, efficacy and cell penetration, effectors of interest will undergo a variety of additional tests as part of the effector suitability and selection process and as part of the regulatory approval process. Such testing includes those tests typically conducted as part of the development and regulatory approval of a pharmaceutical product, including evaluations of pharmacokinetic properties, pharmacodynamic properties, pharmaceutic properties, toxicology, mutagenicity, reproductive toxicity and the like.

Pharmacokinetic evaluations may include analyses of an effector's absorption, distribution, metabolism and excretion profiles in in vitro cell systems, animals, animal disease models, normal humans and patients. The absorption profile includes the rate of absorption, the maximum plasma concentration achieved, the effect of formulation modifications, the effect of different salt and crystal forms, the effect of food and other medications on absorption and the like. The distribution profile includes the determination of the location and concentration of the effector in the various tissues and fluids of the body, protein binding and the like. The metabolism profile includes the determination of the mechanism by which the effector is metabolized, such as by enzymes of the liver or kidney, a determination of the structure and activity of the metabolites produced, the effect of the effector and metabolites on the metabolism of other drugs, the effect of food and other drugs on the metabolism of the effector and the like. The excretion profile includes the determination of the mechanism and distribution of excretion, such as through the bile or kidney, the clearance rate, the half-life (amount of time needed to clear fifty percent of the plasma level of the administered effector), accumulation, and the like of the effector and its metabolites.

Pharmacodynamic evaluations involve an analysis of an effector's physiological activity. Such an analysis may include an assessment of the duration of activity, dosing regiment and formulation effects on activity, therapeutic threshold (minimum effector plasma concentration needed for activity) and mode of administration effects.

Pharmaceutic evaluations will involve an analysis of an effector's physical properties with respect to formulating the effector. Effector physical properties of interest include solubility, chemical stability, such as the effect of temperature, moisture and light, crystal form (solid), salt form (solid or in solution) and the like, solution stability, effect on solution pH, crystal vs. amorphous solid vs. oil vs. liquid, crystal density, etc. Also the effector formulation properties are evaluated. These properties include, but are not limited to, stability, effect of the crystal form and size on absorption, effect of the salt form on absorption, solid compressibility and malleability, solid flowability, uniformity of crystal size, compatibility with other formulation ingredients, packing density, blend and content uniformity of each formulation.

Toxicology involves determining the toxic and other side effect profile of the effector, its metabolites and its formulation, generally initially in animals and later in humans, to ascertain the potential risks involved in administering the effector. Analyses may include an evaluation of undesirable effects on the central nervous system, cardiovascular system, pulmonary system, gastrointestinal system, renal system, hepatic system, genitourinary system, hematopoietic system, immunologic system and dermal system. The analyses may include determining toxic dose, maximum tolerable dose, agonistic or antagonistic activity against other enzymes, receptors, binding proteins and the like, carcinogenicity, immunogenicity, and the like. Means and methods of toxicological analysis are well known in the art. Descriptions include those found in Principles and Methods of Toxicology (Third Edition, 1994, Ed. A. Wallace Hayes, Raven Press, NY) and Toxicology: The Basic Science of Poisons (by Cassarett and Doull, Fifth Edition, 1996, Ed. Curtis D. Klaassen, McGraw-Hill, NY).

Effector development may also include the study and evaluation of one or more of these attributes in special populations such as pediatrics and geriatrics. In addition, the analyses may also require the evaluation of the effects of gender and ethnicity.

As mentioned above, the final selection of a suitable effector will include testing similar to that performed for any new molecular entity (NME) proposed for human use. A variety of screening strategies may be used. Until a few years ago, it was not possible to predict the absorption and metabolism characteristics of NMEs without conducting appropriate whole animal in vivo studies. However, recent advances in the understanding of the molecular biology and functional specificity of metabolic enzymes and absorption and transport mechanisms have provided a mechanistic basis for gathering absorption and metabolism data utilizing “humanized” in vitro systems. The development and availability of these humanized in vitro systems coupled with advances in analytical instrumentation are speeding the development process. It is increasingly possible to conduct high-throughput pharmacokinetic screening of new molecules. The following description will highlight the in vitro and in vivo methods and techniques that are being applied.

In Vitro Methods

Absorption Assays

The ideal drug candidate should possess good metabolism and absorption characteristics. An early prediction of the oral bioavailability of a series of compounds provides invaluable information regarding the structure activity relationship (SAR). An essential part of selecting compounds with good systemic bioavailability involves an accurate prediction of absorption through the gut. One of the most successfully applied techniques for prediction of absorption in man is the 21-day Caco-2 model. Caco-2 cells are of human origin, derived from human colon cancer cells. When cultured on porous membranes these cells spontaneously differentiate into highly functionalized monolayers that are similar in characteristics to the small intestinal enterocytes (Pinto et al., Biol. Cell 47:323-330, 1983; Hidalgo et al., Gastroenterology 96:736-749, 1989.) Data gathered over the years show this model to be a good predictor of in vivo human intestinal absorption (Artursson et al., Biochem. Biophys. Res. Comm. 175:880-885, 1991.) Though this model provides reasonable estimates of in vivo absorption, its use as a high-throughput screening tool has been challenged by some investigators. The labor-intensive and time-consuming nature of conducting studies with these cells has prompted some researchers to look for viable alternatives such as the fast-growing Madin-Darby Canine Kidney (MDCK) cells (Irvine et al., J. Pharm. Sci. 88(1):28-33, 1999) or the three-day Caco-2 culture model (Chong et al., Pharm. Res. 14 (12):1835-1837, 1997) while others have attempted to automate the Caco-2 absorption assessment methodology (Garberg et al., Pharm. Res. 16(3):441-445, 1999.) Irvine et al. utilized a large number of compounds to evaluate the use of MDCK cells as an alternative to Caco-2 cells for estimating membrane permeability. Overall, a good correlation (r²=0.79) was observed for apparent permeability (Pₐₚₚ) values between the Caco-2 and MDCK cells. Based on their findings these authors suggest MDCK cells to be a practical permeability screening tool for increasing throughput in the early discovery phase. The three-day Caco-2 model for studying the permeability of a compound has also been investigated. These three-day cultures provide reasonable Pₐₚₚ, values for transcellularly transported compounds. The monolayers from three-day cultures, however, are about 4- to 6-fold leakier than the traditional 21 day cultured cells, and the predictive nature of these cells for compounds that are transported paracellularly or by efflux and carrier-mediated mechanisms is not as robust (Yee S, Day W: Applications of Caco-2 Cells in Drug Discovery and Development In Handbook of Drug Metabolism. Woolf, T (Eds), Marcel Dekker, Inc. New York: 508-519.) Garberg and colleagues developed an automated set-up for assessment of in vitro permeability to increase the throughput capacity during the screening phase utilizing the traditional Caco-2 model. This automation was accomplished by the incorporation of a liquid handling system that performed all necessary pipetting steps during the course of the experiment. Based on the results obtained, these authors suggested that utilizing an automatic sample processor can substantially increase the capacity of in vitro absorption screening. Another strategy to accelerate the absorption screening throughput has been investigated (Taylor et al., Pharm. Res. 14(5):572-577, 1997.) These researchers conducted absorption experiments using the Caco-2 model with mixtures of physicochemically diverse compounds and were able to successfully select potential orally bioavailable compounds. The pooling of samples obtained from experiments conducted with a single compound or a mixture of compounds has also been investigated utilizing a mass spectrometer as the analytical tool (McCarthy et al., Pharm. Res. 13(9):S242, 1996).

New higher throughput methods are also being developed to screen NMEs for physicochemical properties such as solubility that could influence NME absorption (Tarbit et al., Curr. Opin. Chem. Biol. 2:411-416, 1998.) Others are developing methods to determine if NMEs are substrates of various intestinal transporters (e.g., p-glycoprotein). Permeability data, coupled with physiochemical and transport data, should enhance the ability to predict absorption in the future and will lead to faster selection of lead candidates with desirable absorption characteristics.

Metabolism Assays

First-pass metabolism by the intestine or liver is another major determinant of in vivo oral bioavailability. Metabolic reactions can be broadly classified into Phase I and Phase II reactions. Phase I reactions create a functional group on the molecule so that it can either be further conjugated by phase II enzymes or excreted upon modification. Most of the oxidative metabolic reactions are carried out by the cytochrome P450 (CYP) enzyme system, a superfamily of heme-containing enzymes predominantly found in the liver. CYP catalyzed metabolism can have significant effects on the overall dispositional characteristics of a molecule which could result in a short half-life, low bioavailability, non-linear kinetics, drug-drug interactions, toxicity, lack of efficacy, and intersubject variability. These defects alone or in combination are the leading cause of failure of drugs in development or in some instances have led to withdrawal of a marketed product.

To address these issues metabolism studies are instituted early in the program. High-throughput in vitro screening methods have been developed for predicting the metabolic stability of an NME, and the potential for drug-drug interactions. The traditional test tube and water bath incubation method for metabolic stability testing has been replaced with the 96-well plate technique that is more amenable to automation. The incubations are typically conducted utilizing pooled human or animal liver microsomes or S-9 fraction, and the reaction is carried out at 37° C. on a reaction block. Following termination of the reaction, the plate is placed directly on the high pressure liquid chromatography (HPLC) autosampler coupled to a mass spectrometer. The use of human hepatocytes in a 96-well format has also been proposed (Li A., AAPS, New Orleans, La., 1999.) Sample pooling from a 96-well plate system along with liquid chromatography mass spectrometry (LC/MS) for separation and detection and automated data acquisition has also been used to increase the throughput in the generation of metabolic stability data (Michelson et al., LC-MS Analysis of the Metabolic Stability of Human Liver Microsome Samples Prepared in a 96-well Format. AAPS Pharmsci. 1(4):S-300, 1999.)

After data acquisition, an estimate of in vitro half-life (t_{1/2}) or the apparent clearance (Clₐₚₚ) is made based on the rate of depletion of the parent compound (Obach et al., Journal of Pharmacol. Exp. Ther. 283(1):46-58, 1999.) Predictions of in vivo bioavailability can also be made using the Clₐₚₚ, for the species in question. Rank ordering of the screened compounds based on the Clₐₚₚ or the predicted in vivo bioavailability can then be performed. These results can be generated for a large number of compounds in a rapid turnaround manner and provide structure activity relationship insights for improving pharmacokinetic (PK) characteristics by the means of structural modifications.

The prediction of drug-drug interactions is also an integral part of drug development programs. Traditionally, cytochrome P450 mediated drug-drug interaction studies have been conducted utilizing human liver microsomes, and the analysis of samples is accomplished using HPLC separation techniques with UV, or fluorescence detection. Complete chromatographic separation is quite time-consuming and labor-intensive and is therefore not appealing for use in a high-throughput screening setting. Recently, Yin and colleagues (ISSX Proceedings 15:87, 1999) described a rapid throughput method for the determination of CYP isoform activity to evaluate the inhibitory potential of NMEs. This method utilizes human liver microsomes and is performed on a 96-well platform with solid phase extraction and pooling of samples for high-pressure liquid chromatography coupled to mass spectrometry (LC/MS/MS) quantification. Crespi and coworkers (Curr. Opin. Chem. Biol. 2(1):15-19, 1999) have also described microtiter plate-based fluorometric assays for activities of CYP1A2, CYP2C9, CYP2C19, CYP2D6, and CYP3A4, the five major human drug metabolizing CYP enzymes. Radiometric assays to obtain data from in vitro studies for various CYP enzymes have also been reported (Rodrigues et al., Drug Met. Dis. 24:126-136, 1996; Rodrigues et al., Anal. Biochem. 219:309-320, 1994; and Riley R J, Howbrook D: J. Pharmac. Toxicol. Meth. 38:189-193, 1998.) Moody et al. (Xenobiotica, 9(1):53-75, 1999) recently reported the development and application of a fully automated method for the analysis of catalytic activities of major CYP enzymes. This procedure is based on fluorometric and radiometric assays and is performed by a robotic sample processor.

The methods described above could successfully provide a rapid estimation of IC₅₀ values and thus an initial determination of the inhibitory capability of the NME. Further characterization of the inhibitory potential of NMEs may be pursued using classical approaches that utilize established CYP isoform specific probe substrates (Newton et al., Drug Met. Dis. 23(1):154-158, 1995).

Early identification of induction based drug-drug interactions is another important issue. Previously, induction assays required the use of primary cultures of human hepatocytes that meant heavy reliance of such studies on the availability of human tissue. Recent knowledge of the molecular basis for the induction of CYP3A4 via the PXR nuclear receptor has provided new means for exploring induction based drug-drug interactions in a rapid and relatively inexpensive fashion (Lehmann et al., J. Clin. Invest. 102(5):1016-1023, 1998.

High-throughput screening for metabolic stability and drug-drug interactions generates valuable data that can be exploited to develop “ideal” molecules. In order to maximize the benefit of existing information, the data should be organized in a format that is easily searchable and retrievable. To this end, several drug metabolism databases are now commercially available (Erhardt P: Drug Metabolism Data: Past and Present Status, Med. Chem. Res. 8(7/8):400-421, 1998.) A “knowledge-based” database with systematic organization of literature reports on drug interactions is also being developed by Professor Rene Levy's group at the University of Washington, Seattle (Levy R H et al., Metabolic drug interactions, www.apptechsvs.com/drug/.) This object-oriented database design allows the user to retrieve information regarding the involvement of particular CYP isoforms in the metabolism of a drug, and also includes information on other relevant PK parameters to allow an in vitro-in vivo correlation. The parallel use of these databases along with the data generated from high throughput screening studies will speed up the nomination and development process of lead candidates.

In Vivo Pharmacokinetic Assays

Despite successes in the development of in vitro absorption and metabolism assays, in vivo PK studies still play an important role in drug development (Smith D A, van de Waterbeemd H: Pharmacokinetics and metabolism in early discovery, Curr. Opin. Chem. Biol. 3:373-378, 1999.) To obtain the PK parameter values of an NME, the NME is dosed both intravenously and orally to an animal, blood is sampled at various time points and then analyzed by HPLC or gas chromatography (GC) for the compound of interest. The PK parameters (e.g., clearance, volume of distribution, elimination half-life, and oral bioavailability) which describe the absorption and disposition of the NME in a whole animal are calculated from the serum concentration-time profiles. Feedback on the structure-PK relationship is important in directing research and synthetic efforts and information on in vivo exposures is useful in designing in vivo pharmacology/efficacy studies. In vivo PK screening may also be necessary when in vitro metabolism assays are poorly predictive of in vivo kinetics (e.g., when the compound is eliminated predominantly by renal or biliary mechanisms). Increasingly, pharmaceutical scientists are generating in vivo PK data for use in developing and validating models to predict in vivo kinetics from in vitro data and for use in in-silico modeling (Tarbit et al., Curr. Opin. Chem. Biol. 2:411-416, 1998; Bayliss et al., Curr. Opin. Drug. Dis. Dev. 2(1), 20-25, 1999). In recent years, efforts to increase throughput in PK evaluation of NMEs have focused on mixture dosing and sample pooling to minimize bioanalytical workload. HPLC with mass spectrometric detection (LC/MS and LC/MS/MS) has greatly enhanced the ability to monitor more than one compound in a matrix.

Mixture Dosing

Mixture dosing (also referred to as N-in-One, cassette, or cocktail dosing) involves the simultaneous administration of two or more NMEs to the same animal and the assay of the plasma samples by LC/MS. PK parameter values are determined from the resulting concentration-time profiles. Berman et al. (J. Med. Chem. 40(6):827-829, 1997; and Olah et al., Rapid Commun. Mass Spectrom 11:17-23, 1997) reported the successful application of the mixture dosing approach to speed candidate selection during drug discovery. Since then, several publications have reported on the PK of two to 22 compounds dosed simultaneously to mice, rats, dogs, and monkeys by intravenous and oral routes (Bayliss et al., Curr. Opin. Drug. Dis. Dev. 2(1), 20-25, 1999; Allen et al., Pharm. Res. 15(1):93-97, 1998; Frick et al., Pharm. Sci. Tech. Today. 1(1):12-18, 1998; and Gao et al., J. Chromatogr. A828:141-148, 1998). The reports have demonstrated good agreement between PK parameter values obtained from mixture dosing compared to those obtained from traditional approaches. Mixture dosing offers significant cost- and time-savings because fewer animals are studied, less compound is required, and fewer samples are generated compared to traditional methods. An additional advantage of mixture dosing is the ability to examine tissue distribution (e.g., brain-blood ratios), urinary excretion patterns (Frick et al., Pharm. Sci. Tech. Today. 1(1): 12-18, 1998) and protein binding (Allen et al., Pharm. Res. 15(1):93-97, 1998) of multiple NMEs along with the PK studies. Disadvantages of N-in-One dosing include difficulties in developing formulations for dosing, the possibility of increased adverse events in the animal, and method development issues (e.g., the need for increased sensitivity because of lower doses, avoidance of molecular weight redundancies between metabolites and analytes of interest). There is also a concern that drug-drug interactions (e.g., inhibition of metabolism or other routes of elimination of one compound by another) may alter the PK of NMEs. Compound-compound interactions have occurred during oral mixture dosing and during intravenous dosing when a potent cytochrome P450 inhibitor was introduced into a mixture (Olah et al., Rapid Commun. Mass Spectrom. 11: 17-23, 1997). The interactions were identified by inclusion of a reference compound in all mixture dosing sessions. A list of factors to consider when designing mixture dosing experiments to minimize the chance of obtaining false data and a statistical analysis describing the probability that an NME may experience a compound-compound interaction as a function of mixture size are provided in the review by Frick and colleagues (Pharm. Sci. Tech. Today 1(1):12-18, 1998). Future advances in mixture dosing will likely focus on the automation of various steps such as dose solution preparation, MS tuning and data reduction and on the development of robust, flexible chromatography methods (Bayliss et al., Curr. Opin. Drug Dis. Dev. 2(1), 20-25, 1999; Gao et al., J. Chromatogr. A828:141-148, 1998). Future work should also be focused on defining and minimizing the PK risks (e.g., compound-compound interactions) associated with the method and the use of PK data for the development of structure-PK relationships (see for example, Shaffer et al., J Pharm. Sci. 88(3):313-318, 1999 suggesting that structure-pharmacokinetic relationships can be derived for a set of chemical analogs from data generated using a mixture dosing technique.)

Pooling of samples from pharmacokinetic studies to increase bioanalytical throughput.

Several labs have successfully used a plasma-pooling approach to increase bioanalytical and PK throughput (Olah et al., Rapid Commun. Mass Spectrom. 11:17-23, 1997; and Hop et al., J. Pharm. Sci. 87(7):901-903, 1998). In the pooling approach, multiple animals are dosed with individual NMEs, and aliquots of the samples from common time points are pooled and analyzed by LC/MS. The advantage of pooling is that there are fewer samples to analyze and the possibility of in vivo compound-compound interactions is eliminated. The pooling of samples, however, can be very time-consuming and detection limits may be compromised because of sample dilution.

Hop and colleagues (J. Pharm. Sci. 87(7):901-903, 1998) revived a pooling technique that has been used to obtain PK parameters in pediatric patients. For each animal, aliquots of plasma from each time point were pooled in proportion to the time interval it covered to yield just one sample that had a concentration proportional to the area under the curve (AUC). The major advantage of this approach is a significant reduction in the number of samples and bioanalytical workload. The disadvantages are that information about the concentration-time course (Cmax, Tmax, half-life) is no longer available and pooling is very tedious. The unused portion of the plasma sample for promising compounds can always be reanalyzed to obtain the full PK profile and automation would facilitate pooling. The researchers have applied this technique to the analysis of NMEs for a discovery program where AUC after oral dosing was the main concern. The technique could also be used to determine clearance and bioavailability of compounds.

In Vitro Evolution

For purposes of the present invention, in vitro evolution strategies are typically used to evolve an aptamer for an identified effector. The selected and specific aptamer-effector reaction is a useful tool for in vivo applications: e.g., it allows the engineering of constructs which are not naturally found in the cell, and therefore, are not expected to adversely affect normal cell function.

Several in vitro evolution (selection) strategies (Orgel, Proc. R. Soc. London, B 205: 435, 1979) have been used to evolve new nucleic acid catalysts capable of catalyzing a variety of reactions, such as the cleavage and ligation of RNA and DNA (Joyce, Gene, 82:83-87, 1989; Beaudry et al., Science 257:635-641, 1992; Joyce, Scientific American, 267:90-97, 1992; Breaker et al., TIBTECH 12:268, 1994; Bartel et al., Science 261:1411-1418, 1993; Szostak, TIBS 17:89-93, 1993; Kaufmann et al. U.S. Pat. No. 5,814,476; Kumar et al., FASEB J., 9:1183, 1995; Breaker, Curr. Op. Biotech., 7:442, 1996; and Berzal-Herranz et al., Genes & Develop. 6:129, 1992), and new nucleic acids which act as aptamers, such as an ATP aptamer, HIV Rev aptamer, HIV Tat aptamer and others including oligonucleotide effectors (see Ellington and Szostak, Nature 346:818-822, 1990; Famulok and Szostak, Angew. Chem. Int. Ed. Engl. 31:979-988, 1992; Ellington, Current Biology, 4(5):427-429, 1990; Porta and Lizardi, Bio/Technology 131:161-164, 1995; Tang and Breaker, Chemistry & Biology, 4(6):453-459, 1997; Tang and Breaker, RNA, 3:914-925, 1997; Werstuck and Green, Science, 282:296-298, 1998; Tang and Breaker, Nucleic Acids Research 26(18):4214-4221, 1998; Soukup and Breaker, Proc. Natl. Acad. Sci. USA, 96:3584-3589, 1999; and Robertson and Ellington, Nature Biotechnology 17:62-66, 1999). In addition to these, further descriptions of in vitro evolution processes are provided in Innovir U.S. Pat. No. 5,741,679 and U.S. Pat. No. 5,834,186, WO 9843993 (PCT/US98/06231, Yale University), WO 9827104 (PCT/US97/24158, Yale University) and U.S. Pat. No. 5,817,785 (NeXstar Pharmaceuticals).

In a basic form, the process may involve the following steps.

1) A candidate mixture of nucleic acids of differing sequence is prepared. The candidate mixture typically includes nucleic acids having regions of fixed sequences (i.e., each of the members of the candidate mixture contains the same sequences in the same location) and regions of randomized sequences. The fixed sequence regions may be used for a variety of reasons, including: (a) to assist in the amplification steps described below, (b) to mimic a sequence known to bind to the effector, or (c) to enhance the concentration of a given structural arrangement of the nucleic acids in the candidate mixture. The randomized sequences can be totally randomized (i.e., the probability of finding a base at any position being one in four) or only partially randomized (e.g., the probability of finding a base at any location can be selected at any level between 0 and 100 percent).

2) The candidate mixture is contacted with the selected effector under conditions favorable for an interaction between the effector and nucleic acids of the candidate mixture. Under these circumstances, the interaction between the effector and the nucleic acids can be considered as forming aptamer-effector pairs between the effector and those nucleic acids having the strongest affinity for the effector.

3) The nucleic acids with the highest affinity for the effector are partitioned from those nucleic acids with a lesser affinity to the effector. Because only an extremely small number of different molecules (and possibly only one molecule) corresponding to the highest affinity nucleic acid sequences exist in the candidate mixture, it may be desirable to set the partitioning criteria so that a significant amount of the nucleic acids in the initial candidate mixture (approximately 5-50%) are retained during partitioning.

4) Those nucleic acids selected during partitioning as having relatively higher affinity to the effector are then amplified to create a new candidate mixture that is enriched in nucleic acids having a relatively higher affinity for the effector.

5) By repeating these partitioning and amplifying steps, the newly formed candidate mixture contains fewer and fewer unique sequences, and the average degree of affinity of the nucleic acids to the effector will generally increase. With repeated cycles, the process yields a candidate mixture containing one or more unique nucleic acids representing those nucleic acids from the original candidate mixture having the highest affinity to the effector.

There is no known set number of bases required for the interaction of an aptamer and an effector. A variety of nucleic acid primary, secondary and tertiary structures are known to exist. The structures or motifs that have been shown most commonly to be involved in non-Watson-Crick type interactions are referred to as hairpin loops, symmetric and asymmetric bulges, psuedoknots and myriad combinations of the same. Research suggests that they can be formed in a nucleic acid sequence of less than 30 nucleotides. For this reason, it is often preferred that in vitro evolution procedures with contiguous randomized segments be initiated with nucleic acid sequences containing a randomized segment of about 20 to about 50 nucleotides. Thus, the aptamer will typically contain about 20 to about 50 nucleotides, or more preferably about 20 to about 30 nucleotides, evolved for binding to a specific effector. The small size of the molecule is advantageous for cell delivery as compared with conventional expression regulation constructs which are much larger in size.

It will be appreciated by persons skilled in the art that in vitro evolution techniques may be used to create and identify separate aptamers which may then be used as modular units with catalytic structures for the construction of a variety of different allosteric control modules. Alternatively, the allosteric control modules may be evolved as a single unit with separate regions or domains including an aptamer or effector-binding domain, a catalytic domain, and in some embodiments additional domains including, but not limited to, a target RNA recognition domain and a substrate domain.

It will also be appreciated that for any given aptamer, large combinatorial libraries of effectors (e.g., organic compounds, peptides, small molecules, etc.) produced by chemical synthesis can be evaluated for aptamer binding. Phage display libraries may also be screened for the presence of an effector to bind a selected aptamer. Thus, in another embodiment of the present invention the aptamer may be identified and the effector selected through a screening process.

Catalytic Domain

The other main component of the allosteric control module is the catalytic domain. As described above, in vitro evolution may also be used to evolve new nucleic acid catalysts capable of catalyzing a variety of reactions, such as cleavage, ligation and splicing. In addition, the catalytic domain is a highly specific construct, with the specificity of activity depending not only on the base-pairing mechanism of binding, but also on the mechanism by which the molecule affects the expression of the RNA to which it binds. For example, if inhibition is caused by cleavage of the RNA target, specificity is defined as the ratio of the rate of cleavage of the targeted RNA over the rate of cleavage of non-targeted RNA.

Gene of Interest

The present invention contemplates, in one embodiment, the provision of a desired gene that encodes a protein that is defective or missing from a target cell genome in a patient. The present invention also contemplates a method of treating a patient suffering from a disease state by providing the patient with human cells genetically engineered to encode a required protein. Yet another embodiment is the delivery of a gene to correct a genetic defect. In each of these embodiments of the present invention, the gene of interest is delivered to the recipient cell with an allosteric control module which has been designed to alter the expression of the gene in response to the presence or absence of an identified effector.

Exemplary uses of constructs of the present invention include gene therapy for hereditary diseases. These diseases include, but are not limited to: familial hypercholesterolemia or type II hyperlipidemias (LDL receptor), familial lipoprotein lipase deficiency or type I hyperlipidemias (lipoprotein lipase), phenylketonuria (phenylalanine hydroxylase), urea cycle deficiency (ornithine transcarbamylase), von Gierke's disease (e.g., glycogen storage disease, type I; glucose-6-phosphotases), alpha 1-antitrypsin deficiency (alpha 1-antitrypsin), cystic fibrosis (cystic fibrosis transmembrane conductant regulator), von Willebrand's disease and hemophilia A (Factor VIII), hemophilia B (Factor IX), sickle cell anemia (beta globin), beta thalassemias (beta globin), alpha thalassemias (alpha globin), hereditary sperocytosis (spectrin), severe combined immune deficiency (adenosine deaminase), Duchenne muscular dystrophy (dystrophin minigene), Lesch-Nyhan syndrome (hypoxanthine guanine phosphoribosyl transferase), Gaucher's disease (beta-glucocerebrosidase), Nieman-Pick disease (sphingomyelinase), Tay-Sachs disease (lysosomal hexosaminidase), and maple syrup urine disease (branched-chain keto acid dehydrogenase).

Suitable transgenes for use in the present invention further include, but are not limited to, those encoding proteins such as: nerve growth factor (NGF), ciliary neurotrophic factor (CNTF), brain-derived neurotrophic factor (BDNF), neurotrophins 3 and 4/5 (NT-3 and 4/5), glial cell derived neurotrophic factor (GDNF), transforming growth factors (TGF), and acidic and basic fibroblast growth factor (aFGF and bFGF); sequences encoding tyrosine hydroxylase (TH) and aromatic amino acid decarboxylase (AADC); sequences encoding superoxide dismutase (SOD 1 or 2), catalase and glutathione peroxidase; sequences encoding interferons, lymphokines, cytokines and antagonists thereof such as tumor necrosis factor (TNF), CD4 specific antibodies, and TNF or CD4 receptors; sequences encoding GABA receptor isoforms, the GABA synthesizing enzyme glutamic acid decarboxylase (GAD), calcium dependent potassium channels or ATP-sensitive potassium channels; and sequences encoding thymidine kinase, angiostatin, dopamine, blood clotting factors, erythropoietin, the colony stimulating factors G-, GM- and M-CSF, tissue plasminogen activator, human or animal growth hormones, IGF-1, insulin, KGF, leptin, MGDF, multiple drug resistance, osteoprotogerin, VEGF, VEGF-ra, alpha-interferon, beta-interferon, consensus-interferon, IFN-gamma, IL-12, IL-1ra, IL-2, IL4, and TNFbp.

Other genes of interest contemplated by the invention encode pathogens for use as vaccines. Exemplary genes include, but are not limited to, those encoding: HIV-1 and HIV-2 (sequences other than rev and gp160 sequences); human T-lymphotrophic virus types I and II; respiratory syncytial virus; parainfluenza virus types 1-4; measles virus; mumps virus; rubella virus; polio viruses; influenza viruses; non-human influenza viruses (avian, equine, porcine); hepatitis virus types A, B, C, D and E; rotavirus; Norwalk virus; cytomegaloviruses; Epstein-Barr virus; herpes simplex virus types 1 and 2; varicella-zoster virus; human herpes virus type 6; hantavirus; adenoviruses; hepatitis viruses, rabies, foot and mouth disease virus, chlamydia pneumoniae; chlamydia trachomatis; mycoplasma pneumoniae; mycobacterium tuberculosis; atypical mycobacteria; feline leukemia virus; feline immunodeficiency virus; bovine immunodeficiency virus; equine infectious anemia virus; caprine arthritis encephalitis virus; visna virus, infectious mononucleosis; roseola; pneumonia and adult respiratory distress syndrome; upper and lower respiratory tract infections; conjunctivitis; upper and lower respiratory tract infections; genital tract infections; and pneumonia and encephalitis in sheep. The vaccine vectors may be used to generate intracellular immunity if the gene product is cytoplasmic (e.g., if the gene product prevents integration or replication of a virus). Alternatively, extracellular/systemic immunity may be generated if the gene product is expressed on the surface of the cell or is secreted.

A host (especially a human host) may be immunized against a polypeptide of a disease-causing organism by administering to the host an immunity-inducing amount of a vector of the present invention which encodes the polypeptide. Immunization of a human host with a vector of the invention typically involves administration by inoculation of an immunity-inducing dose of the virus by the parenteral route (e.g., by intravenous, intramuscular or subcutaneous injection), by surface scarification or by inoculation into a body cavity. Typically, one or several inoculations of between about 1000 and about 10,000,000 infectious units each, as measured in susceptible human or nonhuman primate cell lines, are sufficient to effect immunization of a human host.

Additional uses of the materials and methods described herein include, but are not limited to:
1. increasing the expression of monoclonal antibodies by hybridoma cells, i.e., cell lines resulting from the fusion of a B-lymphocytes with a myeloma cell lines. Monoclonal antibodies could be produced by growing the hybridoma in tissue culture or in vivo;
2. increasing plant derived products as used in fragrances and perfumes, flavoring compounds or sweeteners e.g. the basic proteins from Thaumatococcus danielli, insecticides, anti-fungal compounds or pesticides;
3. increasing the expression of a polypeptide which is associated with the rate limiting step in bioleaching of metals such as uranium, copper, silver, manganese etc. For example, as carried out by the organism Thiobacillus sp., algae or fungi;
4. increasing the expression of a polypeptide which is associated with the rate limiting step in removal of nitrogen or phosphate or toxic waste minerals from water, e.g., as carried out by Nitrobacter sp. or Acinetobacter sp.;
5. increasing the expression of a polypeptide which is associated with the rate limiting step in stimulating methane production from biological waste, typically from the methanogenic micro-organisms archaebacteria;
6. increasing the expression of a polypeptide which is associated with the rate limiting step in the biodegradation of marine oil spills (e.g., aliphatic hydrocarbons, halogenated aliphatics, halogenated aromatics). In one instance, biodegradation is effected by the conversion of petroleum products to emulsified fatty acids. Bacteria useful in this invention include, but are not restricted to, Archromobacter, Arthrobacter, Flavobacterium, Nocardia, Pseudomonas (e.g. Pseudomonas oleovorans) and Cytophaga. Yeast useful in this invention include, but are not restricted to, Candida (e.g., Candida tropicalis), Rhodotorula, and Trichosporon;
7. increasing the expression of a polypeptide which is associated with the rate limiting step in biodegradation of lignin;
8. increasing the expression of a polypeptide which is associated with the rate limiting step in the biotransformation of steroids and sterols, e.g., by Rhizopus sp., Saccharomyces, Corynebacterium sp.; D sorbitol to L sorbose by Acetobacter suboxydans; racemic mixtures; prochiral substrates; terpenoids; alicyclic and heteroalicylic compounds; antibiotics; aromatic and heterocyclic structures including phthalic acid esters, lignosulfonates, surfactants and dyes; naphthyridines by Penicillium sp.; polynuclear aromatic hydrocarbons; aliphatic hydrocarbons; amino acid and peptides; glucose to fructose; glucose to gluconic acid; raffinose to sucrose and galactose; lactose; and sucrose.

9. increasing the expression of a polypeptide which is associated with the rate limiting step in the production of commercially important enzymes from micro-organisms, e.g., lactase from Aspergillus oryzae, Escherichia coli, Bacillus stearothermophilus;
10. increasing the expression of a polypeptide which is associated with the rate limiting step in the growth of Saccharomyces on molasses; the growth of Candida on spent sulphite liquor; the growth of yeast on higher n-alkanes; the growth of bacteria on higher n-alkanes; the growth of bacteria or yeast on methane or methanol;
11. increasing the expression of a polypeptide which is associated with the rate limiting step in the assimilation of atmospheric nitrogen by e.g., Azotobacteria sp., Rhizobium sp., or Cyanobacteria;
12. increasing the expression of a polypeptide which is associated with the rate limiting step in the production of insecticides from Bacillus sp., e.g., Bacillus thuringiensis;
13. increasing the expression of a polypeptide which is associated with the rate limiting step in the production of insecticides from entomogenous fungi such as Deuteromycetes, e.g., Verticillium lecanii and Hirsutella thompsonii;
14. increasing the expression of a polypeptide which is associated with the rate limiting step in the production of ethanol from cellulosic materials, starch crops, sugar cane, fodder beats, or molasses by, for example, Saccharomyces cerevisiae, S. uvarum, Schizosaccharomyces pombe or Kluyveromyces sp.;
15. increasing the expression of a polypeptide which is associated with the rate limiting step in acetic acid production from ethanol by Acetobacter sp. or Gluconobacter sp., the rate limiting step in the lactic acid production by the family of Lactobacillaceae, the rate limiting step in the citric acid production by Candida sp. or Aspergillus niger using e.g., molasses or starch, the rate limiting step in gluconic acid production by e.g., Pseudomonas sp., Gluconobacter sp., and Acetobacter sp., or the rate limiting step in the production of amino acids by bacteria or fungi;
16. increasing the expression of a enzyme in a cell, which enzyme catalyzes the resolution of racemic mixtures of amino acids;
17. increasing the expression of a polypeptide which is associated with the rate limiting step in the production of extracellular polysaccharides, e.g., by Corynebacterium sp., Pseudomonas sp., or Erwinia tahitica. Other examples include the production of scleroglycan from the fungus Sclerotium sr., pullulan from Aureobasidium pullulans, curdlan from Alcaligeans faecalis, and dextrans from Streptobacterium sp. or Streptocucus sp. Other examples include anionic polysacharides from Arthrobacter viscosus, bacterial alginates from Azotobacter vinelandii, and xanthan from Xanthomonas campestris;
18. increasing the expression of a polypeptide which is associated with the rate limiting step in the production of an antifungal compound (e.g., Griseofulvin) and penicillins from Penicillium sps.;
19. increasing the expression of a polypeptide which is associated with the rate limiting step in the production of antibiotics by fungi, e.g., polyether antibiotics, chloramphenicol, ansamycines, tetracyclines, macrolides, aminoglycosides, clavans, cephalosporins, cephamycins from Streptomyces sp.;
20. increasing the expression of a polypeptide which is associated with the rate limiting step in the production of antitumor substances, e.g., actinomycin D, anthracyclines, and bleomycin from Streptomyces sp.;
21. increasing the expression of a polypeptide which is associated with the rate limiting step in the production of nucleic acids, nucleotides and related compounds, e.g., 5′inosinate (IMP), 5′guanylate (GMP), cAMP by e.g., Brevibacterium ammoniagenes;
22. increasing the expression of a polypeptide which is associated with the rate limiting step in the production of vitamins, e.g., vitamin B12 by Pseudomonas denitrificans, Propionibacterium shermanii, or Rhodopseudomonas protamicus;
23. increasing the expression of a polypeptide which is associated with the rate limiting step in the production of riboflavin by Ashbya gossypii or Bacillus subtilis.;
24. increasing the expression of a polypeptide which is associated with the rate limiting step in the production of ergosterol by yeast, e.g., Saccharomyces cerevisiae;
25. increasing the expression of a polypeptide which is associated with the rate limiting step in the production of ergot alkaloids by Claviceps sp.;
26. increasing the expression of a polypeptide which is associated with the rate limiting step in the production of secondary metabolites useful for selected therapeutic uses in human medicine, e.g., cyclosporin from Trichoderma polysporum;
27. increasing the expression of a polypeptide which is associated with the rate limiting step in the production of secondary products from plant cell cultures, e.g., cinnamic acid derivatives in Coleus blume and shikonins from Lithospermum erythrophizon;
28. increasing the expression of a polypeptide which is associated with the rate limiting step in the production of wine and beer by Saccharomyces sp.;
29. increasing the expression of a polypeptide which is associated with the rate limiting step in the production of yogurt or cheese by Staphylococcus sp., Lactobacillus sp. and Propionibacterium sp.;
30. increasing the expression of a polypeptide which is associated with the rate limiting step in the fermentation of cocoa from Theobroma cacao by fungi and bacteria; and
31. increasing the expression of a polypeptide which is associated with the rate limiting step in the fermentation of coffee beans from Coffea sp. by fungi and bacteria.

The gene of interest, whose expression is associated with a defined physiological or pathological effect within a multicellular organism, may also be a plant gene. The plant gene may encode an agronomically important trait. Examples of agronomically important traits may include, but are not limited to, germination, sprouting, flowering, fruit ripening, salt tolerance, herbicide resistance, pesticide resistance, fungicide resistance, temperature resistance, and growth.

Additionally, in the practice of the invention the gene of interest may be a protozoan gene. Examples of protozoans may include, but are not limited to, a selection from the group consisting of Trypanosoma, Plasmodium, Leishmania, Giardia, Entamoeba, Toxoplasma, Babesia, and Cryptosporidiosis. Moreover, the gene of interest whose expression is associated with a defined physiological or pathological effect within a multicellular organism, may be a helminth gene.

Clearly, the present invention has commercial applications both in the case where the polypeptide itself is commercially or therapeutically important, and in the case where expression of the polypeptide mediates the production of a molecule which is commercially or therapeutically important.

A further use of the allosteric control modules of the present invention is in the creation of conditional knockout and transgenic animals. In conditional knockouts, the targeted gene product is expressed normally in the genetically altered animal and expression is inhibited only in the presence of an effector. In applying the effectors described herein and the allosteric control modules evolved by the methods described herein to conditional knockouts, one desires to evolve allosteric self-cleaving RNAs that are activated by a small molecule effector identified by the inventive methods. In this case, a DNA construct is created which codes for the gene product of interest that has been altered to place an activatable self-cleaving allosteric control module comprising a catalytic domain, in a preferred embodiment, a ribozyme, in an intron or untranslated region of the gene. The choice of insertion site is made through empirical investigation so that the gene product is expressed at normal or near normal levels in the absence of effector, but is completely or nearly completely inhibited in the presence of effector.

Thus, insertion of an allosteric ontrol module of the present invention in place of the native gene through site specific genetic recombination methods in embryonic stem (ES) cells and subsequent micro-injection of the altered ES cells into blastocysts allows for the creation of an altered organism that normally or near normally expresses the gene of interest during development of the genetically altered organism. This allows for the gene product to be present during development, thus eliminating problems of embryonic lethality or developmental compensation. Subsequently, adult animals can be treated with the effector molecule that activates the cis-acting catalytic domain, resulting in degradation of the RNA coding for the targeted gene product.

Alternatively, one could evolve an inhibitable self-splicing intron that processes itself normally in the absence of effector. In the presence of effector, the activity of the self-splicing intron would be inhibited and the intron would remain in the mRNA resulting in inhibition of gene expression. In either case of activation or inhibition, direct assessment of the biological effects of inhibiting expression of the targeted gene product in the adult animal is achieved.

In the case of transgene over expression, one desires to evolve a cis-acting inhibitable self cleaving ribozyme or an activatable self splicing intron in a very similar manner to that described for the application of RMST in a gene therapy approach for human therapeutic applications. In this particular application, the insertion of the altered gene product that contains the cis-acting inhibitable self-cleaving allosteric control module or activatable self-splicing intron could be accomplished through standard methods employed for the creation of transgenic animals. Swanson et al., Annu. Rep. Med. Chem., 29:265-274, 1994; Polites, Int. J. Exp. Pathol., 77(6):257-262, 1996. Alternatively, the altered gene construct could be introduced to adult animals via viral or naked DNA transfer methods, akin to those being contemplated for gene therapy applications. In either case, over expression of the gene of interest would normally be inhibited due the insertion of the desired allosteric control modules as described herein. The subsequent dosing of the animal with the effector would then result in the over expression of the gene product for assessment of functional outcomes.

Cell therapy or ex vivo gene therapy, e.g., implantation of cells containing the DNA constructs of the present invention, is also contemplated. This embodiment would involve implanting cells containing the DNA constructs by which the expression of the gene of interest is then regulated. In order to minimize a potential immunological reaction, it is preferred that the cells be of human origin and produce a human gene of interest. It is envisioned, however, that the vectors may be used to modify heterologous donor cells and xenogeneic cells, as well as autologous cells, for delivery or implantation.

In some cases, vectors may be delivered through implanting into patients certain cells that have been genetically engineered, using methods such as those described herein, to express and secrete the polypeptides, fragments, variants, or derivatives. Such cells may be animal or human cells, and may be derived from the patient's own tissue (autologous) or from another source, either human (allogeneic) or non-human (xenogeneic). Optionally, the cells may be immortalized. In order to further decrease the chance of an immunological response, the cells may be encapsulated to avoid the infiltration of surrounding tissues. The encapsulation materials are typically biocompatible, semi-permeable polymeric enclosures or membranes that allow release of the protein product(s) but prevent destruction of the cells by the patient's immune system or by other detrimental factors from the surrounding tissues.

Techniques for the encapsulation of living cells are known in the art, and the preparation of the encapsulated cells and their implantation in patients may be accomplished without undue experimentation. For example, Baetge et al. (WO 9505452; PCT/US94/09299) describe membrane capsules containing genetically engineered cells for the effective delivery of biologically active molecules. The capsules are biocompatible and are easily retrievable. The capsules encapsulate cells transfected with recombinant DNA molecules comprising DNA sequences coding for biologically active molecules operatively linked to promoters that are not subject to down regulation in vivo upon implantation into a mammalian host. The devices provide for the delivery of the molecules from living cells to specific sites within a recipient. In addition, see U.S. Pat. Nos. 4,892,538, 5,011,472, and 5,106,627. A system for encapsulating living cells is described in PCT Application WO 9110425 of Aebischer et al. See also, PCT Application WO 9110470 of Aebischer et al., Winn et al., Exper. Neurol. 113:322-329, 1991, Aebischer et al., Exper. Neurol. 111:269-275, 1991; and Tresco et al., ASAIO 38:17-23, 1992.

Promoter

Those skilled in the art will appreciate that it may also be beneficial to provide a promoter with the gene of interest as well as one more additional operational control sequences. The choice of promoter or other operational control sequences, however, is not limiting to the selection of effectors, the evolution of aptamers or the construction and use of the allosteric control modules of the present invention.

Promoter regions vary in length and sequence, and can further encompass one or more DNA-binding sites for sequence-specific DNA binding proteins, and/or an enhancer or silencer. The present invention may employ, for example, a CMV promoter or a P5 promoter. Such promoters, as well as mutations thereof, are well known and have been described in the art (see, e.g., Hennighausen et al., EMBO J. 5:1367-1371, 1986; Lehner et al., J. Clin. Microbiol. 29:2494-2502, 1991; Lang et al., Nucleic Acids Res. 20:3287-95, 1992; Srivastava et al., J. Virol. 45:555-564, 1983; and Green et al., J. Virol. 36:79-92, 1980). Other promoters, however, can also be employed, such as the Ad2 or Ad5 major late promoter and tripartite leader, the Rous sarcoma virus (RSV) long terminal repeat, and other constitutive promoters, as have been described in the literature. For instance, the herpes thymidine kinase promoter (Wagner et al., Proc. Natl. Acad. Sci. USA., 78:144-145, 1981), the regulatory sequences of the metallothionine gene (Brinster et al., Nature, 296:39-42, 1982) promoter elements from yeast or other fungi, such as the Gal 4 promoter, the alcohol dehydrogenase promoter, the phosphoglycerol kinase promoter, and the alkaline phosphatase promoter, can be employed. Similarly, promoters isolated from the genome of mammalian cells or from viruses that grow in these cells (e.g., Ad, SV40, CMV, and the like) can be used.

Delivery

The DNA constructs described herein can be incorporated into a variety of vectors for introduction into cells. Suitable vectors include, but are not restricted to, naked DNA, plasmid DNA vectors, viral DNA vectors (such as adenovirus or adeno-associated virus vectors), viral RNA vectors (such as retroviral or alphavirus vectors) (for a review see Couture and Stinchcomb, 1996, supra) and non-viral vectors (such as DNA complexed with cationic lipids or packaged within liposomes). It will be appreciated by those skilled in the art that an expression vector will also include a) a transcription initiation region; b) a transcription termination region, and c) expression control sequences. It will also be appreciated that the DNA constructs and vectors may be produced by joining separately produced components. For example, the promoter, aptamer and catalytic domain may be separately manufactured by chemical synthesis or recombinant DNA/RNA technology and then joined.

Vectors containing the DNA constructs of the present invention may be delivered to cells by a variety of plasmid and non-viral delivery methods known to those familiar with the art, including, but not restricted to, liposome-mediated transfer or lipofection, by incorporation into other delivery vehicles, such as hydrogels, cyclodextrins, biodegradable nanocapsules, and bioadhesive microspheres, naked DNA delivery (direct injection or direct uptake), receptor-mediated transfer (ligand-DNA complex), electroporation, calcium phosphate mediated transformation, microinjection, osmotic shock, microparticle bombardment (e.g., gene gun), bio-chip materials and combinations of the above. Delivery materials and methods may also involve the use of components including, but not limited to, inducible promoters, tissue-specific enhancer-promoters, DNA sequences designed for site-specific integration, DNA sequences capable of providing a selective advantage over the parent cell, labels to identify transformed cells, negative selection systems (safety measures), cell-specific binding agents (for cell targeting), cell-specific internalization factors, transcription factors to enhance expression by a vector as well as methods of vector manufacture. Such additional methods and materials for the practice of gene therapy techniques are described in U.S. Pat. No. 4,970,154 electroporation techniques; WO 9640958 nuclear ligands; U.S. Pat. No. 5,679,559 concerning a lipoprotein-containing system for gene delivery; U.S. Pat. No. 5,676,954 involving liposome carriers; U.S. Pat. No. 5,593,875 concerning methods for calcium phosphate transfection; and U.S. Pat. No. 4,945,050 wherein biologically active particles are propelled at cells at a speed whereby the particles penetrate the surface of the cells and become incorporated into the interior of the cells.

A typical use of the constructs of the present invention involves the transfer of a vector to a recipient cell. The recipient cell or host cell is typically a prokaryotic cell in protein production techniques and is preferably a eukaryotic cell in gene therapy techniques. The eukaryotic host cell can be modified either in vitro or in vivo. According to the invention, “contacting” of cells with the vectors of the present invention can be by any means by which the vectors will be introduced into the cell. In one preferred embodiment, viral vectors will be introduced by infection using the natural capability of the virus to enter cells (e.g., the capability of adenovirus to enter cells via receptor-mediated endocytosis). The viral and plasmid vectors, however, can be introduced by any suitable means.

Suitable viral vectors include, but are not limited to, retrovirus, adenovirus, herpes simplex virus, lentivirus, hepatitis virus, parvovirus, papovavirus, poxvirus, alphavirus, coronavirus, rhabdovirus, paramyxovirus, and papilloma virus vectors. U.S. Pat. No. 5,672,344 describes an in vivo viral-mediated gene transfer system involving a recombinant neurotrophic HSV-1 vector. U.S. Pat. No. 5,399,346 provides examples of a process for providing a patient with a therapeutic protein by the delivery of human cells which have been treated in vitro to insert a DNA segment encoding a therapeutic protein. Additional methods and materials for the practice of gene therapy techniques are described in U.S. Pat. No. 5,631,236 involving adenoviral vectors; U.S. Pat. No. 5,672,510 involving retroviral vectors; and U.S. Pat. No. 5,635,399 involving retroviral vectors expressing cytokines.

Preferably, the vectors persist in the cells to which they are delivered. Alternatively, some vectors may be used that provide for transient expression of the DNA constructs. Such vectors might be repeatedly administered as necessary.

Compositions and Administration

Host cells may be transformed with the vectors of the present invention either in vivo or in vitro. If in vitro, the desired target cell type may be removed from the subject, recombinantly modified by vector delivery, and reintroduced into the subject. The modified cells can be screened for those cells harboring the gene of interest, using conventional techniques such as Southern blots and/or PCR. Such modified cells may be transplanted directly into the subject or may be placed in a device which is implanted in the subject. It is also envisioned that the ex vivo modified cells may include non-human cell lines for direct or indirect implantation.

If delivered in vivo, the vector may be formulated into a pharmaceutical composition. The vector may be administered parenterally, e.g., by injection intravenously, intraperitoneally, intramuscularly, intrathecally or subcutaneously. Additional vector formulations suitable for other modes of administration include oral (including sublingual and buccal) and pulmonary (intranasal and inhalation) formulations, topical and transdermal formulations, and suppositories. Delivery may involve a single dose schedule or a multiple dose schedule.

For example, the intramuscular injection of a vector of the present invention, such as a rAAV particles containing the DNA construct, may provide efficient transduction of postmitotic muscle fibers and prolonged transgene expression. According to the invention, this is accomplished without significant inflammation or activation of immunity to the transgene product. Muscle is also particularly well suited for the production of secreted therapeutic protein, such as factor IX or apolipoprotein (Apo) E, among other genes of interest.

When administered by injection, it will be appreciated by those skilled in the art that the vectors of the present invention are typically suspended in a biologically compatible solution or pharmaceutically acceptable delivery vehicle. One such suitable and common vehicle is sterile saline. Other aqueous and non-aqueous isotonic sterile injection solutions and aqueous and non-aqueous sterile suspensions may also be employed and are well known as suitable pharmaceutically acceptable carriers in the art.

In one embodiment of the present invention the vectors contain a gene encoding a therapeutic protein. The vectors are administered in sufficient amounts to provide sufficient levels of expression of the selected protein such that a therapeutic benefit may be obtained without undue adverse effects and with medically acceptable physiological effects which can be determined by those skilled in the medical arts. Dosages of the vector will depend primarily on factors such as the condition being treated, the selected transgene, the age, weight and health of the patient, and thus, may vary among patients. For example, a therapeutically effective dose of the vector of the present invention may be in the range of from about 1 to about 50 ml of saline solution containing concentrations of from about 1×10⁸ to 1×10¹¹ particles/ml rAAV virions containing the DNA constructs of the present invention. A more preferred human dosage may be about 1-20 ml saline solution at the above concentrations. The levels of expression of the selected gene can be monitored to determine the selection, adjustment or frequency of administration. Administration of the vector might then be repeated as needed.

When practiced in vivo, any suitable organs or tissues or component cells can be targeted for vector delivery. Preferably, the organs/tissues/cells employed are of the circulatory system (i.e., heart, blood vessels or blood), respiratory system (i.e., nose, pharynx, larynx, trachea, bronchi, bronchioles, lungs), gastrointestinal system (i.e., mouth, pharynx, esophagus, stomach, intestines, salivary glands, pancreas, liver, gallbladder), urinary system (i.e., kidneys, ureters, urinary bladder, urethra), nervous system (i.e., brain and spinal cord, and special sense organs such as the eye) and integumentary system (i.e., skin). Even more preferably the cells being targeted are selected from the group consisting of heart, blood vessel, lung, liver, gallbladder, urinary bladder, and eye cells.

Accordingly, the present invention also provides a method of obtaining stable gene expression in a host, or modulating gene expression in a host, which comprises administering the vectors of the present invention using any of the aforementioned formulations and routes of administration or alternative routes known to those skilled in the art and appropriate for a particular application. The “effective amount” of the vector or pharmaceutical composition is such as to produce the desired effect in a host which can be monitored using several end-points known to those skilled in the art. For example, effective nucleic acid transfer to a host cell could be monitored in terms of a therapeutic effect (e.g. alleviation of some symptom associated with the disease or syndrome being treated), or by further evidence of the transferred gene or coding sequence or its expression within the host (e.g., using the polymerase chain reaction, Northern or Southern hybridizations, or transcription assays to detect the nucleic acid in host cells, or using immunoblot analysis, antibody-mediated detection, or particularized assays to detect protein or polypeptide encoded by the transferred nucleic acid, or impacted in level or function due to such transfer). One such particularized assay includes an assay for expression of a reporter or marker gene.

While it will be appreciated that the constructs of the present invention may be used with any vector system, a preferred system involving the use of rAAV particles is described as an exemplary system for the following descriptions of pharmaceutical compositions. Such compositions may comprise a therapeutically effective amount of an rAAV particle product in admixture with a pharmaceutically acceptable agent such as a pharmaceutically acceptable carrier. An exemplary carrier material may be water for injection, preferably supplemented with other materials common in solutions for administration to mammals. Typically, an rAAV particle therapeutic compound will be administered in the form of a composition comprising particles in conjunction with one or more physiologically acceptable agents. Neutral buffered saline or saline mixed with serum albumin are exemplary appropriate carriers. Other standard pharmaceutically acceptable agents may be included as desired. For example, other compositions may comprise a buffer or preservative.

The rAAV particle pharmaceutical compositions typically include a therapeutically or prophylactically effective amount of rAAV particles in admixture with one or more pharmaceutically and physiologically acceptable formulation agents selected for suitability with the mode of administration. Suitable formulation materials or pharmaceutically acceptable agents include, but are not limited to, antioxidants, preservatives, diluting agents, suspending agents, solvents, fillers, bulking agents, buffers, delivery vehicles, and diluents. For example, a suitable vehicle may be water for injection, physiological saline solution, or artificial cerebrospinal fluid, possibly supplemented with other materials common in compositions for parenteral administration. Neutral buffered saline or saline mixed with serum albumin are further exemplary vehicles. The term “pharmaceutically acceptable carrier” or “physiologically acceptable carrier” as used herein refers to a formulation agent(s) suitable for accomplishing or enhancing the delivery of the rAVV particles as a pharmaceutical composition.

The primary solvent in a composition may be either aqueous or non-aqueous in nature. In addition, the vehicle may contain other formulation materials for modifying or maintaining the pH, osmolarity, viscosity, clarity, color, sterility, stability, rate of dissolution, or odor of the formulation. Similarly, the composition may contain additional formulation materials for modifying or maintaining the rate of release of rAVV particles, or for promoting the absorption or penetration of rAVV particles.

When systemically administered, the therapeutic compositions for use in this invention may be in the form of a pyrogen-free, parenterally acceptable aqueous solution. The preparation of such pharmaceutically acceptable solutions, with due regard to pH, isotonicity, stability and the like, is within the skill of the art.

Therapeutic formulations of rAVV particles compositions useful for practicing the present invention may be prepared for storage by mixing the selected composition having the desired degree of purity with optional physiologically acceptable stabilizers (Remington's Pharmaceutical Sciences, 18th Edition, A. R. Gennaro, ed., Mack Publishing Company, 1990). Acceptable stabilizers preferably are nontoxic to recipients and are preferably inert at the dosages and concentrations employed, and preferably include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

The optimal pharmaceutical formulation will be determined by one skilled in the art depending upon the intended route of administration, delivery format and desired dosage. See for example, Remington's Pharmaceutical Sciences, 18th Ed. (Mack Publishing Co., Easton, Pa. 18042 pages 1435-1712, 1990.)

An effective amount of an rAVV particle composition to be employed therapeutically will depend, for example, upon the therapeutic objectives such as the indication for which the gene delivered by the rAVV particle is being used, the route of administration, and the condition of the patient. Accordingly, it may be necessary for the therapist to titer the dosage and modify the route of administration as required to obtain the optimal therapeutic effect. Typically, a clinician will administer the composition until a transgene dosage is reached that achieves the desired effect. The composition may therefore be administered as a single dose, or as two or more doses (which may or may not contain the same amount of rAAV particles) over time, or as a continuous infusion via implantation device or catheter.

As further studies are conducted, information will emerge regarding appropriate dosage levels for the treatment of various conditions in various patients, and the ordinary skilled worker, considering the therapeutic context, the type of disorder under treatment, the age and general health of the recipient, will be able to ascertain proper dosing.

A particularly suitable vehicle for parenteral injection is sterile distilled water in which the rAVV particle composition is formulated as a sterile, isotonic solution, properly preserved. Yet another preparation may involve the formulation of rAVV particles with an agent, such as injectable microspheres, bio-erodible particles or beads, or liposomes, that provides for the controlled or sustained release of the product which may then be delivered as a depot injection.

The preparations of the present invention may include other components, for example parenterally acceptable preservatives, tonicity agents, cosolvents, wetting agents, complexing agents, buffering agents, antimicrobials, antioxidants and surfactants, as are well known in the art. For example, suitable tonicity enhancing agents include alkali metal halides (preferably sodium or potassium chloride), mannitol, sorbitol and the like. Suitable preservatives include, but are not limited to, benzalkonium chloride, thimerosal, phenethyl alcohol, methylparaben, propylparaben, chlorhexidine, sorbic acid and the like. Hydrogen peroxide may also be used as preservative. Suitable cosolvents are for example glycerin, propylene glycol and polyethylene glycol. Suitable complexing agents are for example caffeine, polyvinylpyrrolidone, beta-cyclodextrin or hydroxypropyl-beta-cyclodextrin. Suitable surfactants or wetting agents include sorbitan esters, polysorbates such as polysorbate 80, tromethamine, lecithin, cholesterol, tyloxapal and the like. The buffers can be conventional buffers such as borate, citrate, phosphate, bicarbonate, or Tris-HCl.

The formulation components are present in concentrations that are acceptable to the site of administration. For example, buffers are used to maintain the composition at physiological pH or at slightly lower pH, typically within a pH range of from about 5 to about 8.

A pharmaceutical composition may be formulated for inhalation. For example, rAVV particles may be formulated as a dry powder for inhalation. Alternatively, rAVV particle inhalation solutions may be formulated in a liquefied propellant for aerosol delivery. In yet another formulation, solutions may be nebulized.

Additional rAVV particle formulations will be evident to those skilled in the art, including formulations involving rAVV particles in combination with one or more other therapeutic agents. Techniques for formulating a variety of other sustained- or controlled-delivery means, such as liposome carriers, bio-erodible microparticles or porous beads and depot injections, are also known to those skilled in the art.

Regardless of the manner of administration, the specific dose may be calculated by the delivery of a gene that produces a therapeutic effect according to body weight, body surface area or organ size. Further refinement of the calculations necessary to determine the appropriate dosage for treatment involving each of the above mentioned formulations is routinely made by those of ordinary skill in the art and is within the ambit of tasks routinely performed by them. Appropriate dosages may be ascertained through use of appropriate dose-response data.

The route of administration of the composition is in accord with known methods, e.g., inhalation, injection or infusion by intravenous, intraperitoneal, intracerebral (intraparenchymal), intracerebroventricular, intramuscular, intraocular, intraarterial, or intralesional routes, or by sustained release systems which may optionally involve the use of a catheter.

In this regard, the cell to be transformed will depend on the purpose for gene transfer; for example, the disease state being treated. For example, the rAAV particle can be used to deliver selected nucleotide sequences into any nucleated cell including stem, progenitor and erythroid cells; as well as any of the various white blood cells such as lymphocytes, neutrophils, eosinophils, basophils, monocytes; tissue specific cells, such as those derived from lung, heart, kidney, liver, spleen, pancreatic tissue, connective tissue, bone tissue including osteocytes, gangliocytes, epithelial and endothelial cells, ependymal cells, reticuloendothelial cells, dendritic and neural cells, skeletal muscle, cardiac muscle and smooth muscle cells, and the like. It is further envisioned that the constructs of the present invention are useful in the delivery of a gene of interest to tumor cells and pathogen infected cells. AAV has been reported to infect all established cell lines thus far examined.

It will also be appreciated that the same dosage calculations and considerations of routes of administration and pharmaceutical formulations are applicable to the effector used to alter the expression of the gene of interest.

### EXAMPLES

The following non-limiting examples further illustrate the identification and selection methods as well as the synthesis and use of exemplary constructs of the present invention. Those of ordinary skill will recognize that these are non-limiting examples and that the present invention discloses a means to provide a broad array of other effectors, allosteric control modules and vectors which can be used as described herein for the regulation of gene expression.

### Example 1

### Effector Identification

A method for identifying an effector for use in the evolution of aptamers and the alteration of gene expression involves the following steps. The first step is the selection a set of desired characteristics for an effector, wherein the desired characteristics include one or more of the following:
a) at least 1% bioavailability;
b) biodistribution to tissue containing an allosteric control module;
c) the ability to pass to the nucleus of the cell;
d) either no drug interactions or manageable drug interactions;
e) either no toxicity or acceptable toxicity at the dosage range used;
f) either no side effects or acceptable side effects at the dosage range used;
g) either no pharmacological effect at the dosage range used in regulating transgene expression or a negligible pharmacological effect; and
h) physical properties suitable for the in vitro evolution of an aptamer.
   The selected characteristics indicate that the effector is suitable for aptamer generation, human consumption and use with an allosteric control module for the alteration of gene expression. Second, one or more databases containing information on the selected effector characteristics is accessed to evaluate these attributes and a set of effectors having the selected characteristics is identified. The effectors may then be used to generate aptamers by means of in vitro evolution.

Physical properties which will further aid in the identification of a suitable effector for the in vitro evolution of an aptamer include, but are not limited to the following attributes.
sufficient structural and functional group complexity to provide interactive sites for the identification of high affinity aptamers; preferably the molecules will be rigid, planar entities
solubility in an aqueous solution at a level which allows molecular evolution techniques to be performed
appropriate charge, i.e., lack of an excessive number of ionizable groups that would lead to unfavorable interactions with nucleic acid ligands
   Thus, highly flexible, lipophilic molecules are not favored.

For purposes of the present invention, manageable drug-drug interactions are interactions that can be avoided by avoiding concomitant use of the drugs or reduced through a combination of monitoring and dose adjustments. For purposes of the present invention, acceptable toxicity at the dosage range used includes the use of the effector at a dose which is not toxic or which has reversible toxicity or is acceptable in view of desired treatment. For purposes of the present invention, acceptable side effects at the dosage range used includes side effects which abate or end with continued use and those which are managed by other means (for example, prevented or suppressed by the use of other drugs, diet, etc.)

Using databases, such as those described above, the following molecules were identified for evaluation as potential effectors:

| | |
|---|---|
| 1) Atevirdine/nonnucleoside reverse | Pharmacia & Upjohn |
| transcriptase inhibitor | |
| Development Status | Phase III; discontinued/more potent |
| | successor |
| Bioavailability: | Oral; well absorbed orally, 30-60 minutes |
| | to peak serum |
| Biodistribution: | Widely distributed, crosses blood-brain |
| | barrier and placenta; maximum |
| | concentration of drug in serum (oral |
| | delivery): 7.3 uM |
| Intracellular Localization: | Intracellular target (HIV RT) |
| Toxicity/Drug Interactions: | Well tolerated (tested up to 1600 mg oral |
| | delivery); no effects on vital signs, ECG |
| | or lab tests; buffering interferes with |
| | absorption; rash in some symptomatic |
| | HIV patients |
| Other Pharmacology: | Viral target, no other known activity |
| Target Localization: | Intracellular (HIV RT) |
| Functionality for Aptamer: | Hydrophobic, basic, large surface |
| Synthesis/Scale Up: | Produced in multi-ton quantities |
| Further described in: | WO91/9849 1991, priority US 457483 |
| Structure: | 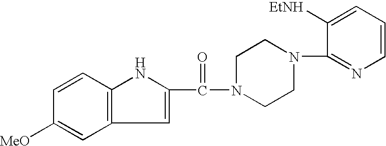 |
| 2) Talviraline/nonnucleoside reverse | Bayer/Hoechst Marion/Glaxo-Wellcome |
| transcriptase inhibitor | (BAY-10-8979, HBY-097) |
| Development Status: | Phase II; discontinued/more potent |
| | successor |
| Bioavailability: | Oral absorption (375-3000 mg/day in |
| | Phase II) |
| Biodistribution: | Wide |
| Intracellular Localization: | Intracellular Target (HIV RT) |
| Toxicity: | Well tolerated (rash in two patients who |
| | received dosing three times daily) |
| Other Pharmacology: | Viral target, no other known activity |
| Target Localization: | Intracellular (HIV RT) |
| Functionality for Aptamer: | Hydrophobic, basic |
| Synthesis/Scale Up: | Produced in sufficient quantities for 180 |
| | patient Phase II clinical trial |
| Structure: | 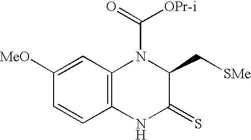 |
| 3) Quinolones/DNA topoisomerase | multiple sources |
| inhibitors | |
| Development Status: | Phase II, III, active |
| Bioavailability: | Oral |
| Biodistribution: | Wide/muscle |
| Toxicity: | Acceptable/phototoxicity |
| Other Pharmacology: | Antibacterial |
| Target Localization: | Intracellular/intrabacterial |
| Name(s): | Fandofloxacin, DW-116 |
| Structure: | 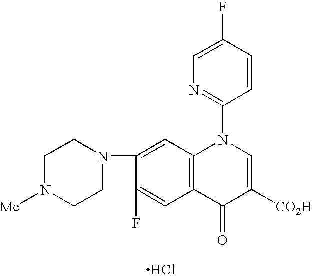 |
| Development status: | Dong Wa, Phase II complete, development |
| ongoing | |
| Criteria: | |
| Bioavailability: | Orally absorbed |
| Biodistribution: | Good organ distribution including muscle |
| Intracellular Localization: | Intracellular, intrabacterial |
| Toxicity/Drug Interactions: | Well tolerated, v. low phototoxicity |
| Other Pharmacological Activity: | Anti-infective, anti-microbial |
| Functionality for Aptamer: | Basic, hydrophobic |
| Synthesis/Scale Up: | Sufficient for Phase II in Europe and Korea |
| Further description: | U.S. 5,496,947 |
| Name(s): | Bay-y-3118 |
| Structure: | 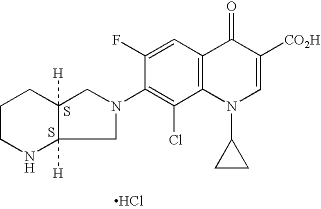 |
| Development status: | Bayer, Phase II 1993, patent on more potent |
| analogs | |
| Criteria: | |
| Bioavailability: | Orally absorbed (Tmax 1.3 Hrs, Half life |
| 11.4 Hrs | |
| Intracellular Localization: | Intracellular, intrabactenal |
| Toxicity/Drug Interactions: | No abnormal findings in humans |
| Other Pharmacological Activity: | Anti-infective, anti-microbial |
| Functionality for Aptamer: | Basic, hydrophobic |
| Synthesis/Scale Up: | Phase II supplied |
| Further description: | EP 520240 |
| 4) Vitamin D3 analogs | Leo |
| Development Status: | Phase II, III, active |
| Bioavailability: | Low Oral (Form. Diff.)/transdermal |
| Biodistribution: | Highly potent/side effects common |
| Toxicity: | Hypercalcemia |
| Other Pharmacology: | Vitamin D receptor |
| Target Localization: | Intracellular (nuclear receptor) |
| 5) MCC-555/peroxisome proliferation | Johnson & Johnson/Mitsubishi |
| activated receptor | |
| Development Status: | Phase II; active/diabetes |
| Bioavailability: | Oral |
| Biodistribution: | Muscle and adipose |
| Toxicity: | Liver toxicity (controversial) |
| Other Pharmacology: | Peroxisome proliferation activated |
| | receptor/hypoglycaemia |
| Target Localization: | Nuclear |
| 6) GW-2570/peroxisome proliferation | Glaxo |
| activated receptor | |
| Development Status: | Phase II; active/diabetes |
| Bioavailability: | Oral |
| Biodistribution: | Muscle and adipose |
| Toxicity: | Unknown |
| Other Pharmacology: | Peroxisonie proliferation activated |
| | receptor/hypoglycaemia |
| Target Localization: | Nuclear; high stereoselectivity for active |
| | component |
| 7) Triclabendazole | Novartis |
| Development Status: | Phase II; third world, veterinary (Fasinex) |
| Bioavailability: | Oral |
| Biodistribution: | Liver/metabolism important |
| Toxicity: | Low/none in humans |
| Other Pharmacology: | antihelmintic/flukecide |
| Target Localization: | Unknown |
| Structure | 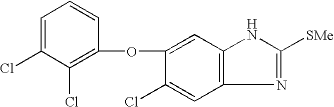 |

Currently, the preferred effectors include the normucleoside reverse transcriptase inhibitors. These compounds have good oral bioavailability, are nontoxic and have no known activity other than against the viral target.

### Example 2

### In Vitro Evolution

An exemplary in vitro evolution strategy is described as follows. A random pool of nucleic acids is synthesized wherein, each member contains two portions: a) one portion consists of a region with a defined (known) nucleotide sequence; b) the second portion consists of a region with a degenerate (random) sequence. The known nucleotide sequences may provide several advantages/uses. For example, a certain nucleotide sequence may be known or expected to bind to a given effector. Alternatively, the known sequence may facilitate or provide for complementary DNA (cDNA) synthesis and PCR amplification of molecules selected for their effector binding. In yet another aspect, the sequences may be used to introduce a restriction endonuclease site for the purpose of cloning. The random sequence can be created to be completely random (each of the four nucleotides represented at every position within the random region) or the degeneracy can be partial (Beaudry and Joyce, 1992 supra) and involve the use of rational design. Sequence variation in test nucleic acids can be introduced or increased by mutagenesis before or during the selection/amplification iterations. This random library of nucleic acids is incubated under conditions that ensure folding of the nucleic acids into conformations that facilitate the desired activity (e.g., effector binding and catalysis). Following incubation, nucleic acids are converted into complementary DNA (if the starting pool of nucleic acids is RNA).

Nucleic acids with desired trait may be separated or partitioned from the rest of the population of nucleic acids by a variety of methods. For example, a filter-binding assay can be used to separate the fraction that binds the desired effector from those that do not. The fraction of the population that is bound by the effector (for example) may be the population that is desired (active pool). Typically, the binding of the effector and the RNA is assessed by applying the RNA pool or mixture to an affinity matrix containing the effector with which the aptamer will specifically bind or react. The non-binding RNA species are removed or washed away, and the specifically-binding species are eluted from the effector for further use in the evolution process. A new piece of DNA (containing new oligonucleotide primer binding sites for PCR and restriction sites for cloning) may be introduced to the termini of molecules in the active pool (to reduce the chances of contamination from previous cycles of selection) to facilitate PCR amplification and subsequent cycles (if necessary) of evolution.

Amplification is preferably performed by means of reverse transcription of the eluted species into DNA followed by polymerase chain reaction. The result of the amplification process is the production of a large number of the selected RNA-encoding DNA molecules. The final pool of nucleic acids with the desired trait (i.e., aptamer(s) which bind to the effector) may be cloned into a plasmid vector and transformed into bacterial hosts. Recombinant plasmids can then be isolated from transformed bacteria, and the identity of clones can be determined using DNA sequencing techniques.

Multiple cycles of selection and amplification should result in the selective enrichment of RNA species that bind to the effector tightly and specifically. Cycles of selection and amplification are repeated until a desired goal is achieved. With the current techniques and materials, the cycles are typically repeated five or more times. In the most general case, the evolutionary steps of selection and amplification may be continued until no significant improvement in binding strength of RNA to effector is achieved upon repetition of a cycle.

In some cases, however, it is not necessarily desirable to repeat the iterative steps of in vitro evolution until a single RNA is identified. The RNA pool may include a family of nucleic acid structures or motifs that have a number of conserved sequences and a number of sequences which can be substituted or added without significantly effecting the affinity of the nucleic acid molecules to the effector. By terminating the process prior to the identification of a single aptamer, it is possible to determine the sequence of a number of suitable RNAs.

To further improve specificity, a negative selection process can also be used. A negative selection procedure may be employed before, during or after the in vitro evolution process. The negative selection provides the ability to discriminate between closely related but different effectors. Thus, negative selection can be introduced to identify aptamers that have a high specificity for an effector but do not recognize either other members of the effector's family or other structurally similar molecules. For example, in the evolution of an aptamer for theophylline, caffeine may be used to counter-select and eliminate those aptamers which would cross-react with the structurally similar molecule caffeine.

One post-evolution method would be to perform a negative selection on a pool that has already been evolved against the desired effector. The process would involve the use of either an effector family member or a molecule structurally similar to the desired effector as the negative selection target. The selected population is passed over an affinity column containing the negative selection target and those nucleic acids which bind to the negative selection target are removed from the selected pool. Alternatively, the selected population may be passed over an affinity column containing the desired effector, and the pool is then challenged by the addition of a negative selection target. Preferably, this process would also involve the performance of two to three negative selections using the negative selection target and a late-round, highly evolved pool that was evolved using the effector. The binding of certain sequences to the negative selection target would be used to subtract those sequences from the evolved pool. This method allows one to quickly eliminate from several hundred to several thousand nucleic acid sequences that demonstrate a high affinity for both the effector and molecules having similar structural characteristics.

It will be appreciated that “separating” or “partitioning” may include any process for separating the selected RNAs from the remainder of the unreacted RNA candidate mixture. Separation can be accomplished by various methods known in the art. Filter binding, size separation, affinity chromatography, liquid-liquid partitioning, filtration, gel shift, density gradient centrifugation are all examples of suitable partitioning methods. Separation may also be accomplished by means of the presence of a PCR priming site that remains on the catalytically inactive RNA. Equilibrium partitioning methods can also be used. The simple partitioning methods include any method for separating a solid from a liquid, such as, centrifugation with and without oils, membrane separations and simply washing. The RNAs may also be specifically eluted from the effector with a specific antibody or ligand. The choice of partitioning method will depend on properties of the effector and the RNA and can be made according to principles and properties known to those of ordinary skill in the art.

The amplification process may be any process or combination of process steps that increases the amount or number of copies of a molecule or class of molecules. In preferred embodiments, amplification occurs after members of the test mixture have been partitioned, and it is the facilitating nucleic acid associated with a desirable product that is amplified. For example, the amplification of RNA molecules can be carried out by a sequence of three reactions: the use of reverse transcription to make cDNA copies of selected RNAs, the use of the polymerase chain reaction to increase the copy number of each cDNA, and the transcription of the cDNA copies to obtain RNA molecules having the same sequences as the selected RNAs. Any reaction or combination of reactions known in the art can be used as appropriate, including direct DNA replication, direct RNA amplification and the like, as will be recognized by those skilled in the art. The amplification method should result in the proportions of the amplified mixture being essentially representative of the proportions of different sequences in the mixture prior to amplification.

### Example 3

### Allosteric Control Module

The allosteric control module contains a catalytic domain and an aptamer, evolved as described above, that is selected such that the interaction of the aptamer with an effector alters the activity of the allosteric control module. The interaction of the effector and aptamer may result in an alteration of the catalytic activity of the catalytic domain. Depending upon the selection of the components, the alteration can result in either an increase or a decrease in the activity of the catalytic domain of the module.

Ribozymes, ribozyme-like molecules and portions of such molecules are used to form the catalytic domains of the present invention. Ribozymes which are useful in the present invention include, but are not limited to, molecules in the classes of hammerhead, axehead, hairpin, hepatitis delta virus, neurospora, self-splicing introns (including group I and group II), newt satellite ribozymes, Tetrahymena ribozymes, ligases, peptide ligases, phosphatases and polymerases. The nucleic acids of these molecules may be used or the molecules may be used as the starting point for the production of ribozyme-like, synthetic, non-naturally occurring sequences.

The allosteric control module may include additional components or domains including a substrate domain (for example, in the case of self-cleaving catalytic domains) or a recognition domain (to aid in the recognition of the site at which catalytic activity is to be directed). The allosteric control module may also be designed such that the catalytic domain and aptamer are joined by a structural bridge, wherein the interaction of the aptamer and effector results in an alteration of the bridge which in turn results in an alteration of catalytic activity of the catalytic domain (see Soukup and Breaker, Proc. Nat. Acad. Sci. USA, 96:3584-3589, 1999).

The allosteric control module is then tested in vitro and/or in vivo. The optimal constructs in an effector-activated expression control system will provide the maximum inhibition of expression in the absence of effector and the maximum enhancement of expression in the presence of effector. In an effector-inactivated expression control system, the optimal constructs will provide the maximum inhibition of expression in the presence of effector and the maximum enhancement of expression in the absence of effector.

While not being limiting to the present invention, the catalytic domain may be synthesized by procedures for normal chemical synthesis of RNA as described in Usman et al., J. Am. Chem. Soc. 109:7845, 1987; Scaringe et al., Nucleic Acids Res. 18:5433, 1990; and Wincott et al., Nucleic Acids Res. 23:2677-2684, 1995. The details will not be repeated here, but such procedures may involve the use of common nucleic acid protecting and coupling groups, such as dimethoxytrityl at the 5′end, and phosphoramidites at the 3′-end.

In addition, the catalytic activity of the molecules can be optimized as described by Draper et al., PCT WO93/23569, and Sullivan et al., PCT WO94/02595; Ohkawa et al., Nucleic Acids Symp. Ser. 27:15-6, 1992; Taira et al., Nucleic Acids Res. 19:5125-30, 1991; Ventura et al., Nucleic Acids Res. 21:3249-55, 1993; and Chowrira et al., J. Biol. Chem. 269:25856, 1994. The details will not be repeated here, but include altering the length of the ribozyme binding arms, or chemically synthesizing ribozymes with modifications (base, sugar and/or phosphate) that prevent their degradation by serum ribonucleases and/or enhance their enzymatic activity (see e.g., Eckstein et al., International Publication No. WO 9207065; Perrault et al., Nature 344, 565, 1990; Pieken et al., Science 253, 314, 1991; Usman and Cedergren, Trends in Biochem. Sci. 17, 334, 1992; Usman et al., International Publication No. WO 9315187; and Rossi et al., International Publication No. WO 9103162; and Sproat, U.S. Pat. No. 5,334,711; all of these describe various chemical modifications that can be made to the base, phosphate and/or sugar moieties of enzymatic RNA molecules). Modifications which enhance their efficacy in cells, and removal of bases from stem loop structures to shorten RNA synthesis times and reduce chemical requirements may be used.

### Example 4

### Aptamer Evolution and Selection

One embodiment of the present invention involves effector identification in accordance with Example 1 together with an evolution and selection of an aptamer. The aptamer evolution first involves preparing a pool or mixture of random sequence single-stranded RNA (ssRNA) with constant regions that are necessary for reverse transcription and PCR amplifications. Typically the individual ssRNAs contain at least 20 nucleotides, but fewer nucleotides may be used. The mixture of ssRNA is then contacted with an effector. Next, the RNAs which bind to the effector are separated from the remainder of RNAs in the pool which do not bind to the effector. Those separated RNAs are amplified to form DNA, and the amplified DNA is used to form an enriched mixture of RNAs which bind to the effector. The effector-recognition, partitioning and amplification steps are performed for one or more cycles as needed to identify one or more of the RNAs as an aptamer(s) which best bind the effector.

The evolved aptamer or aptamers are then used in an allosteric control module. The selection of the aptamer for use in an allosteric control module is performed by first linking the aptamer to a catalytic RNA to form an allosteric control module; and then identifying those allosteric control modules in which the interaction of the effector and aptamer alters the activity of the catalytic RNA. This analysis may be performed in vivo or in vitro.

### Example 5

### In Vitro Aptamer Evolution and Selection for Theophylline

This example describes the evolution of an allosteric regulatable hammerhead ribozyme comprising an aptamer specific for theophylline. Theophylline was chosen as an effector for the reason, among others, that it has been approved for use by the FDA since 1940 and its safety and toxicity profiles are acceptable and well-known. Jenison et al. previously characterized a theophylline-binding aptamer, Science 263:1425-1429, 1994, to which Zimmerman et al., assigned its secondary structure. Nature Struct. Biol. 4:644-649, 1997. The two RNA strands that connect the hammerhead catalytic domain to the theophylline aptamer domain, the so-called “communication module,” contains only six nucleotides and was completely randomized. The complexity of this library is 1.7×10⁶ individual molecules.

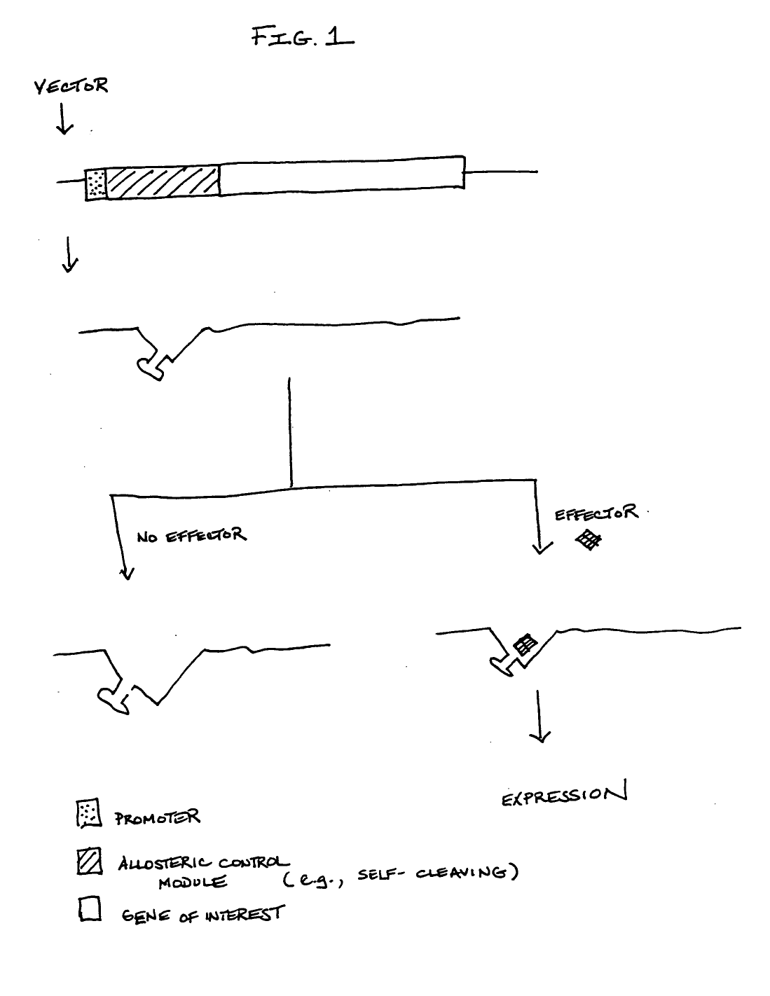

Selection was initiated with 2 nmoles of synthetic single-stranded DNA Template 1 (SEQ ID NO: 1; FIG. 1) which was made into its double stranded form by the polymerase chain reaction (PCR).

DNA Template 1
5′ GGGAGAGGGA TCCAGCTGAC GANNNNNNAA TACCAGCCGA
AAGGCCCTTG GCAGGNNNNN NGAAACGCCT
TCGGCGTCCT GGAT 3′

Five rounds of PCR were carried out in 500 μL reaction volume containing 50 mM KCl, 10 mM Tris-HCl (pH 9.0 at 25° C.), 0.01% TritonX-100, 2.5 mM EDTA, 0.25 mM DNTP each, and 2 nmoles of the two primers, Forward T7-11:
5′ GCTTAATACGACTCA CTATAGGGAGAGGGATCCAGC 3′ (SEQ ID NO: 2)
   and Reverse T7-11:
5′ GAAGTTCTAATC CAGGACGCCGAAGGCGTT′T 3′ (SEQ ID NO: 3).

Parameters for each PCR cycle were 30 sec. at 93° C., 30 sec. at 55° C., and 1 min at 72° C. Resulting double-stranded DNA containing a priming site for T7 RNA polymerase template was concentrated by ethanol precipitation. Approximately 25% of DNA was used for in vitro transcription carried out at 37° C. overnight in 500 μL reaction volume containing 40 mM Tris-HCl (pH 8.0 at 25° C.), 20 mM MgCl₂, 2 mM Spermidine (Sigma, St. Louis, Mo.), 0.01% Triton X-100, 5 mM DTT, 400 U of T7 RNA polymerase and 4 mM NTP each. DNA template in the post-transcription mixture was removed by a brief DNAse I treatment (20 U/500 μL) at 37° C. for 15 min. The ribozyme library containing the random sequence region in the Stem-II was isolated by running transcribed RNA on an 8% polyacrylamide gel under denaturing conditions (denaturing PAGE).

Ribozymes that did not cleave in response to Theophylline were removed by incubating the RNA in a ribozyme cleavage buffer (RZCL buffer: 50 mM Tris-HCl (pH 7.5 at 25° C.) and 10 mM MgCl₂) at 25° C., punctuated at 30 min intervals by incubation at 85° C. for 30 sec. This negative selection was carried out for 5 cycles of thermal punctuation. The RNA population resistant to ligand-independent self-cleavage was isolated by denaturing PAGE. A positive selection of the ligand-independent cleavage resistant RNA population was carried out by incubating this RNA in RZCL buffer containing 200 μM Theophylline at 25° C. for 5 min to facilitate Theophylline-dependent cleavage of ribozymes. The resulting population of 5′ fragments upon self-cleavage was isolated by denaturing PAGE. The 5′-fragment RNA population was subsequently used as the template for reverse transcription in 30 μL reaction volume containing 50 mM KCl, 50 mM Tris-HCl (pH 8.0 at 25° C.), 5 mM MgCl₂, 5 mM DTT, 1 mM DNTP each, 10 U of avian myeloblastosis virus reverse transcriptase, and 500 pmoles of Reverse T7-11 primer at 42° C. for 30 min. The resulting cDNA was used as the template for PCR to obtain the template to generate RNA for the next round of in vitro selection.

We carried out seven cycles of selections, during which the concentration of Theophylline and MgCl₂ in the positive selection step was gradually decreased from 200-20 mM and 10-2 mM, respectively. After seven cycles, the enriched population exhibited theophylline-dependent self-cleavage activity. The resultant PCR products were purified by gel filtration and ethanol precipitation. Purified PCR products (0.1 pmoles) were cloned into pT7Blue-3 Vector (Novagen, Madison, Wis.) in EcoRV site with Perfectly Blunt Cloning Kit (Novagen) according to the protocol supplied by the manufacturer. The ligation mixture was transformed into NovaBlue Singles Competent Cells (Novagen) using standard protocols. Plasmids in the transformants were isolated and sequenced with U 19 vector-based primer (SEQ ID NO: 4) (5′ GTTTTCCCAGTCACGACGT 3′) and subjected to further analysis.

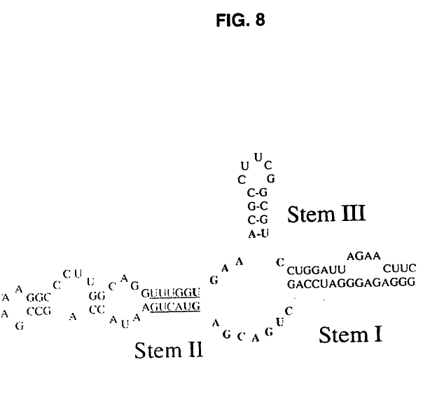

An example of an individual ribozyme sequence, AT-50, that responds to Theophylline for cleavage is illustrated in FIG. 8 and has the following sequence (SEQ ID NO:5):

5′ GGGAGAGGGA UCCAGCUGAC GAGUACUGAA UACCAGCCGA
AAGGCCCUUG GCAGGUUUGG UGAAACGCCU UCGGCGUCCU
GGAUUAGAAC UUC 3′

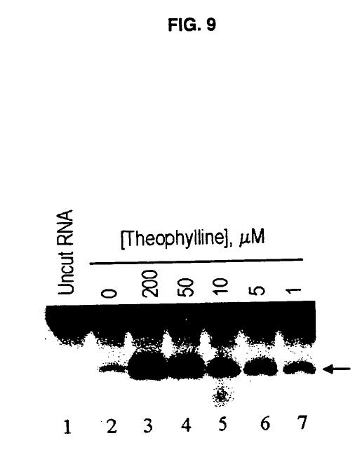

The self-cleaving ability of ribozyme TA-50 is strongly dependent on the presence of theophylline (FIG. 9). The highest cleavage activity of TA-50 ribozyme was observed with theophylline concentration between 10-50 μM. Since this concentration of theophylline is within the therapeutically achievable range, it is possible to envision an in vivo application of TA-50 or another ribozyme with similar performance characteristics to modulate gene expression in response to theophylline intake.

### Example 6

### Effector Inactivation of an Allosteric Control Module

The following procedure produces allosteric control modules having a catalytic activity which is inactivated or inhibited in the presence of the identified effector. Effector identification is performed in accordance with Example 1 followed by the evolution and selection of an aptamer which involves the steps of:
a) preparing a pool of random sequence ssRNA wherein each ssRNA comprises an aptamer, a proposed catalytic domain and one or more constant regions suitable for reverse transcription and PCR amplification;
b) identifying those RNAs which have catalytic activity;
c) amplifying the catalytically active RNAs to form coding DNA molecules;
d) transcribing the amplified DNA to form an enriched mixture of catalytically active RNA;
e) contacting the mixture with an effector;
f) selecting those RNAs which bind to the effector but which do not retain catalytic activity upon binding the effector;
g) amplifying the selected RNAs to form coding DNA molecules;
h) transcribing the amplified DNA to form an enriched mixture of allosteric control modules having a catalytic activity which is inactivated or inhibited in the presence of the effector; and
i) performing steps b) through h) for one or more cycles as needed to identify one or more allosteric control modules which recognize, bind and interact with the effector and which are inactivated or inhibited by effector binding when the effector and the selected allosteric control module are used in the modulation of gene expression.

In an alternative embodiment, the evolution of the allosteric control module, involves the steps of:
a) preparing a pool of random sequence ssRNA wherein each ssRNA comprises an aptamer, a proposed catalytic domain and one or more constant regions suitable for reverse transcription and PCR amplification;
b) contacting the mixture with an effector;
c) selecting those RNAs which bind to the effector but which do not demonstrate catalytic activity upon binding the effector;
d) amplifying the selected RNAs to form DNA molecules;
e) transcribing the amplified DNA to form a RNA mixture;
f) selecting those RNA as one or more allosteric control modules which demonstrate catalytic activity in the absence of the effector;
g) amplifying the selected RNAs;
h) transcribing the amplified RNA to form an enriched mixture of allosteric control modules having a catalytic activity which is inactivated or inhibited in the presence of the effector; and
i) performing steps b) through h) for one or more cycles as needed to identify one or more allosteric control modules which recognize, bind and interact with the effector and which are inactivated or inhibited by effector binding when the effector and the selected allosteric control module are used in the modulation of gene expression.

In one embodiment the “catalytic activity” is a self-cleaving activity and the self-cleaving allosteric control module is used for the inhibition or reduction the expression of a gene of interest in the absence of the effector. In further embodiments, the catalytic activity involves ligase activity or splicing activity.

### Example 7

### Effector Activation of an Allosteric Control Module

The following procedure produces allosteric control modules having a catalytic activity which is activated in the presence of the identified effector. Effector identification is performed in accordance with Example 1 followed by the evolution and selection of an aptamer which involves the steps of:
a) preparing a mixture of random sequence ssRNA wherein each ssRNA comprises an aptamer, a proposed catalytic domain and one or more constant regions suitable for reverse transcription and PCR amplification;
b) identifying those RNAs which do not demonstrate catalytic activity in the absence of effector;
c) amplifying the identified RNAs to form coding DNA molecules;
d) transcribing the amplified DNA to form an enriched mixture of RNA;
e) contacting the mixture with an effector;
f) identifying those RNAs which bind to the effector and demonstrate catalytic activity upon binding the effector;
g) amplifying the identified RNAs to form coding DNA molecules;
h) transcribing the amplified DNA to form an enriched mixture of allosteric control modules having a catalytic activity which is activated in the presence of effector; and
i) performing steps b) through h) for one or more cycles as needed to identify one or more allosteric control modules which recognize, bind and interact with the effector and which are activated or enhanced by effector binding when the effector and the selected allosteric control module are used in the modulation of gene expression.

In an alternative embodiment, the evolution of the allosteric control module, involves the steps of:
a) preparing a pool of random sequence ssRNA wherein each ssRNA comprises an aptamer, a proposed catalytic domain and one or more constant regions suitable for reverse transcription and PCR amplification;
b) contacting the pool with an effector;
c) identifying those RNAs which bind to the effector and demonstrate catalytic activity while bound to the effector;
d) amplifying the identified RNAs to form coding DNA molecules;
e) transcribing the amplified DNA to form an enriched mixture of RNA having a catalytic activity in the presence of effector;
f) selecting those RNA which are catalytically inactive in the absence of effector;
g) amplifying the selected RNAs to form coding DNA molecules;
h) transcribing the amplified DNA to form an enriched mixture of allosteric control modules having a catalytic activity which is activated in the presence of effector; and
i) performing steps b) through h) for one or more cycles as needed to identify one or more allosteric control modules which recognize, bind and interact with the effector and which are activated or enhanced by effector binding when the effector and the selected allosteric control module are used in the modulation of gene expression.

In one embodiment the “catalytic activity” is a self-cleaving activity and the self-cleaving of the allosteric control module results in the formation of a functional mRNA encoding a gene of interest. In further embodiments, the catalytic activity involves ligase activity or splicing activity.

### Example 8

### Alternate Effector Selection Procedure

An alternate method of selecting an effector, involves the steps of:
a) providing an allosteric control module suitable for use in the modulation of gene expression;
b) contacting the allosteric control module with one or more effectors; and
c) determining whether or not the interaction of the allosteric control module and an effector results in an alteration of the catalytic activity of the allosteric control module.

Another method of determining whether a molecule not previously known to be an effector may be used in combination with an allosteric control module to specifically alter the expression of a gene of interest involves the steps of:
(a) contacting a sample which contains a predefined number of eucaryotic cells with the molecule to be tested, each cell comprising a DNA construct encoding,
   i) an allosteric control module, and
   ii) a reporter gene that produces a detectable signal, coupled to, and under the control of, a promoter, under conditions wherein the molecule if capable of acting as a modulator of the gene of interest, causes a detectable signal to be produced by the reporter gene;
(b) quantitatively determining the amount of the signal produced in (a);
(c) comparing the amount of signal determined in (b) with the amount of signal produced and detected in the absence of any molecule being tested or with the amount of signal produced and detected upon contacting the sample in (a) with other molecules, thereby identifying the test molecule as an effector which causes a change in the amount of detectable signal produced by the reporter gene, and thereby determining whether the test molecule specifically alters expression of the gene of interest.

### Example 9

### Inducible Gene Expression

The Figures are schematics of several specific embodiments of this technology to regulate expression of genes. The system's utility can be extended beyond direct medical applications into more basic research applications, diagnostic applications and environmental testing applications.

In one embodiment, one can select for an effector-responsive allosteric control module and gene of interest to be introduced into the cell by means of a viral or nonviral vector. Where the allosteric control module involves a self-cleaving catalytic domain, the interaction of the effector and allosteric control module results in the expression of the gene of interest. In the absence of the effector, the mRNA is untranslatable. Upon removal of effector from the system the allosteric control module should return to the active conformation and the expression levels decrease due to the presence of untranslatable mRNA.

In an alternative embodiment, one can select for an effector-responsive allosteric control module and gene of interest to be introduced into the cell by means of a viral or nonviral vector. Where the allosteric control module involves a self-cleaving catalytic domain, the interaction of the effector and allosteric control module may be designed to result in the inactivation of the mRNA and the non-expression of the gene of interest in the presence of effector. In the absence of the effector, the mRNA is translatable.

In another embodiment, one can select for an effector-responsive allosteric control module and gene of interest to be introduced into the cell by means of a viral or nonviral vector. Where the allosteric control module involves a self-splicing catalytic domain, the interaction of the effector and allosteric control module results in the expression of the gene of interest. In the absence of the effector, the mRNA is untranslatable.

In an alternative embodiment, one can select for an effector-responsive allosteric control module and gene of interest to be introduced into the cell by means of a viral or nonviral vector. Where the allosteric control module involves a self-splicing catalytic domain, the interaction of the effector and allosteric control module may be designed to result in the inactivation of the mRNA and the non-expression of the gene of interest. In the absence of the effector, the mRNA is translatable.

It will be appreciated that the DNA constructs may include a regulatable allosteric control module (e.g., self-cleaving format) placed under the control of a constitutive promoter. The same construct may include the coding region for the gene of interest, a stop codon and a poly(A) tail. The introduction of the vector into a cell will result in the production of mRNA encoding both the allosteric control module and gene. If the allosteric control module is active in the absence of effector, then the mRNA will be cleaved by the catalytic domain to yield pieces available for exonucleolytic attack; that is, mRNA will be inactivated. When the effector is administered and enters the cells, the allosteric control module becomes inactive and the mRNA will be translated normally. Hence, gene expression will be under the inducible control of the allosteric control module.

All of the references cited herein, including patents, patent applications, and publications, are hereby incorporated in their entireties by reference.

While this invention has been described with an emphasis upon preferred embodiments, it will be apparent to those of ordinary skill in the art that variations in the preferred embodiments can he prepared and used and that the invention can be practiced otherwise than as specifically described herein. The present invention is intended to include such variations and alternative practices.

Accordingly, this invention includes all modifications encompassed within the spirit and scope of the invention as defined by the following claims.

1. A method for identifying an effector and generating an interactive aptamer or aptamers, said method comprising the steps of:
a) selecting a set of desired characteristics for an effector, wherein the desired characteristics are selected from the group consisting of:
(i) at least 1% bioavailability;
(ii) biodistribution to tissue containing an allosteric control module;
(iii) the ability to pass to the nucleus of the cell;
(iv) either no drug interactions or manageable drug interactions;
(v) either no toxicity or acceptable toxicity at the dosage range used;
(vi) either no side effects or acceptable side effects at the dosage range used;
(vii) either no pharmacological effect at the dosage range used in regulating transgene expression or a negligible pharmacological effect; and
(viii) physical properties suitable for the in vitro evolution of an aptamer;
wherein said characteristics indicate that the effector is suitable for aptamer generation, human consumption and use with an allosteric control module for the regulation of transgene expression;
b) accessing one or more databases containing data on the selected effector characteristics;
c) identifying a set of effectors having said selected characteristics; and
d) generating and selecting aptamers to the effectors in said set by means of in vitro evolution.

2. A method of claim 1, wherein said effector is selected from the group consisting of small organic molecules, peptides, polypeptides, proteins, oligonucleotides, polynucleotides, nucleic acids, naturally occurring metabolites and biological effectors, lipids, carbohydrates (polysaccharides, sugar), fatty acids, and polymers.

3. A method of claim 1, wherein the evolution and selection of said aptamer comprises the steps of:
a) preparing a pool of random sequence single-stranded RNA (ssRNA) each comprising at least 20 nucleotides with constant regions that are necessary for reverse transcription and PCR amplifications;
b) contacting the pool of ssRNA with an effector;
c) separating the RNAs which bind to the effector from the remainder of the pool which does not bind to the effector;
d) amplifying those separated RNAs which bind to the effector to form DNA;
e) transcribing the amplified DNA to form an enriched RNA mixture;
f) performing steps b) through e) for one or more cycles as needed to identify one or more RNAs as one or more aptamers which best bind said effector; and
g) selecting said identified aptamer or aptamers for use in an allosteric control module.

4. A method of claim 1, wherein the random sequence single-stranded RNA each comprise at most 200 nucleotides with constant regions that are necessary for reverse transcription and PCR amplifications.

5. A method of claim 3, wherein selecting said aptamer for use in an allosteric control module comprises the steps of:
a) linking said aptamer to a catalytic RNA to form an allosteric control module; and
b) identifying those allosteric control modules in which the interaction of the effector and aptamer alters the activity of said catalytic RNA in vivo.

6. A method of claim 1, further comprising the selection of said allosteric control module, wherein said method comprises the steps of:
a) preparing a pool of random sequence ssRNA wherein each ssRNA comprises an aptamer, a proposed catalytic domain and one or more constant regions suitable for reverse transcription and PCR amplification;
b) identifying those RNAs which have catalytic activity;
c) amplifying the catalytically active RNAs to form coding DNA molecules;
d) transcribing the amplified DNA to form an enriched mixture of catalytically active RNA;
e) contacting the mixture with an effector;
f) selecting those RNAs which bind to the effector but which do not retain catalytic activity upon binding the effector;
g) amplifying the selected RNAs to form coding DNA molecules;
h) transcribing the amplified DNA to form an enriched mixture of allosteric control modules having a catalytic activity which is inactivated or inhibited in the presence of said effector; and
i) performing steps b) through h) for one or more cycles as needed to identify one or more allosteric control modules which recognize, bind and interact with said effector and which are inactivated or inhibited by effector binding when said effector and said selected allosteric control module are used in the modulation of gene expression.

7. A method of claim 1, further comprising the selection of said allosteric control module, wherein said method comprises the steps of:
a) preparing a pool of random sequence ssRNA wherein each ssRNA comprises an aptamer, a proposed catalytic domain and one or more constant regions suitable for reverse transcription and PCR amplification;
b) contacting the pool with an effector;
c) selecting those RNAs which bind to the effector but which do not demonstrate catalytic activity upon binding the effector;
d) amplifying the selected RNAs to form DNA molecules;
e) transcribing the amplified DNA to form a RNA mixture;
f) selecting those RNA as one or more allosteric control modules which demonstrate catalytic activity in the absence of said effector;
g) amplifying the selected RNAs;
h) transcribing the amplified RNA to form an enriched mixture of allosteric control modules having a catalytic activity which is inactivated or inhibited in the presence of said effector; and
i) performing steps b) through h) for one or more cycles as needed to identify one or more allosteric control modules which recognize, bind and interact with said effector and which are inactivated or inhibited by effector binding when said effector and said selected allosteric control module are used in the modulation of gene expression.

8. A method of claim 6 or 7, wherein said catalytic activity is a self-cleaving activity and wherein the self-cleaving allosteric control module is used for the inhibition or reduction the expression of a gene of interest in the absence of the effector.

9. A method of claim 6 or 7, wherein said ssRNA comprise at least 20 nucleotides.

10. A method of claim 1, further comprising the selection of said allosteric control module, wherein said method comprises the steps of:
a) preparing a pool of random sequence ssRNA wherein each ssRNA comprises an aptamer, a proposed catalytic domain and one or more constant regions suitable for reverse transcription and PCR amplification;
b) identifying those RNAs which do not demonstrate catalytic activity in the absence of effector;
c) amplifying the identified RNAs to form coding DNA molecules;
d) transcribing the amplified DNA to form an enriched mixture of RNA;
e) contacting the mixture with an effector;
f) identifying those RNAs which bind to the effector and demonstrate catalytic activity upon binding the effector;
g) amplifying the identified RNAs to form coding DNA molecules;
h) transcribing the amplified DNA to form an enriched mixture of allosteric control modules having a catalytic activity which is activated in the presence of effector; and
i) performing steps b) through h) for one or more cycles as needed to identify one or more allosteric control modules which recognize, bind and interact with said effector and which are activated or enhanced by effector binding when said effector and said selected allosteric control module are used in the modulation of gene expression.

11. A method of claim 1, further comprising the selection of said allosteric control module, wherein said method comprises the steps of:
a) preparing a pool of random sequence ssRNA wherein each ssRNA comprises an aptamer, a proposed catalytic domain and one or more constant regions suitable for reverse transcription and PCR amplification;
b) contacting the pool with an effector;
c) identifying those RNAs which bind to the effector and demonstrate catalytic activity while bound to the effector;
d) amplifying the identified RNAs to form coding DNA molecules;
e) transcribing the amplified DNA to form an enriched mixture of RNA having a catalytic activity in the presence of effector;
f) selecting those RNA which are catalytically inactive in the absence of effector;
g) amplifying the selected RNAs to form coding DNA molecules;
h) transcribing the amplified DNA to form an enriched mixture of allosteric control modules having a catalytic activity which is activated in the presence of effector; and
i) performing steps b) through h) for one or more cycles as needed to identify one or more allosteric control modules which recognize, bind and interact with said effector and which are activated or enhanced by effector binding when said effector and said selected allosteric control module are used in the modulation of gene expression.

12. A method of claim 11, wherein said ssRNA comprise at least 20 nucleotides.

13. A method of claim 10 or 11, wherein said catalytic activity is a self-splicing activity and wherein the self-splicing of the allosteric control module results in the formation of a functional mRNA encoding a gene of interest.

14. A method of selecting an effector, comprising the steps of:
a) providing an allosteric control module suitable for use in the modulation of gene expression;
b) contacting said allosteric control module with one or more effectors; and
c) determining whether or not the interaction of said allosteric control module and an effector results in an alteration of the catalytic activity of said allosteric control module.

15. A method of claim 1 wherein said databases contain data selected from the group consisting of:
a) marketed drugs with stereoselectivity for an isomer that comprises the pharmaceutically active component and another isomer with little or no pharmacological activity;
b) known drug metabolites having little or no activity;
c) nuclear receptor targeted molecules;
d) drug candidates which entered clinical trials, but the trials were discontinued due to a relative lack of efficacy;
e) drugs that were removed from the market because of lack of efficacy;
f) drugs that are efficacious but which are not marketed because of low relative benefit;
g) drugs designed as antiviral/anti-infectives, for use in patients not affected by the targeted virus or infectious agent;
h) well characterized food additives;
i) generic drugs with well known mechanisms of action; and
j) drugs that were displaced from the market or clinical trials by best in class molecules.

16. A method of claim 1, wherein said database is selected from the group consisting of Investigational Drugs database, Drug Data Report, World Drug Index, Derwent Drug File, R&D Insight, R&D Focus, Pharmaprojects, MEDLINE and EMBASE.

17. A method of determining whether a molecule not previously known to be an effector may be used in combination with an allosteric control module to specifically alter the expression of a gene of interest which comprises:
(a) contacting a sample which contains a predefined number of eucaryotic cells with the molecule to be tested, each cell comprising a DNA construct encoding,
i) an allosteric control module, and
ii) a reporter gene that produces a detectable signal, coupled to, and under the control of, a promoter,
under conditions wherein the molecule if capable of acting as a modulator of the gene of interest, causes a detectable signal to be produced by the reporter gene;
(b) quantitatively determining the amount of the signal produced in (a);
(c) comparing the amount of signal determined in (b) with the amount of signal produced and detected in the absence of any molecule being tested or with the amount of signal produced and detected upon contacting the sample in (a) with other molecules, thereby identifying the test molecule as an effector which causes a change in the amount of detectable signal produced by the reporter gene, and thereby determining whether the test molecule specifically alters expression of the gene of interest.

18. A DNA construct comprising:
(a) a DNA encoding a promoter;
(b) a DNA encoding a desired product; and
(c) a DNA encoding an allosteric control module of claim 1 wherein the catalytic activity of said allosteric control module is altered by the binding of an effector thereto.

19. A host cell comprising the DNA construct of claim 18.

20. A RNA comprising a nucleotide sequence encoding:
(a) a 5′ untranslated region (UTR), one or more introns and a 3′ UTR;
(b) a desired product; and
(c) an allosteric control module of claim 1 wherein the catalytic activity of said allosteric control module is altered by the binding of an effector thereto.

21. A method of modulating the in vivo expression of a desired product in a cell, comprising:
a) providing the cell with a DNA construct of claim 18;
b) introducing into the cell an effector which alters the catalytic activity of said allosteric control module.

22. A packaging cell line for the production of a recombinant viral vector, the cell line containing a viral vector construct comprising a DNA construct of claim 18.

23. A recombinant viral vector comprising a DNA construct of claim 18.

24. The catalytic RNA molecule of claim 1 wherein the catalytic domain comprises a nucleic acid selected from the group consisting of hammerhead ribozyme nucleic acids, axehead ribozyme nucleic acids, hairpin ribozyme nucleic acids, hepatitis delta virus ribozyme nucleic acids, newt satellite ribozyme nucleic acids, Tetrahymena ribozyme nucleic acids, external guide sequences for RNAase P, self-splicing introns, ligases, phosphatases, polymerases and peptide ligases.

25. The catalytic RNA molecule of claim 1 wherein the catalytic RNA is an external guide sequence for RNAase P.

26. The catalytic RNA molecule of claim 1 wherein the catalytic RNA is inactivated when the effector is bound to the aptamer.

27. The catalytic RNA molecule of claim 1 wherein the catalytic RNA is activated when the effector is bound to the aptamer.

28. The catalytic RNA molecule of claim 1 wherein the effector is exogenously administered to cells containing the allosteric control module and a transgene.

29. A process for preparing an allosteric control module for the regulation of gene expression, which comprises:
(a) screening a random nucleic acid library to select an aptamer binding to a selected effector molecule; and
(b) preparing a nucleic acid comprising a sequence for the selected aptamer and a sequence encoding a protein of interest;
wherein the gene encoding the protein of interest is not expressed when the effector molecule binds to the sequence for the selected aptamer.

30. The process of claim 29, further comprising optimizing the selected aptamer by in vitro evolution.


SEQUENCE LISTING (6 sequences)

SEQ ID NO: 1 - DNA, 84 bp, Artificial
  synthetic construct
gggagagggatccagctgacgannnnnnaataccagccgaaaggcccttggcaggnnnnnngaaacgccttcggcgtcctggat

SEQ ID NO: 2 - DNA, 36 bp, Artificial
  synthetic construct
gcttaatacgactcactatagggagagggatccagc

SEQ ID NO: 3 - DNA, 31 bp, Artificial
  synthetic construct
gaagttctaatccaggacgccgaaggcgttt

SEQ ID NO: 4 - DNA, 19 bp, Artificial
  synthetic construct
gttttcccagtcacgacgt

SEQ ID NO: 5 - RNA, 93 nt, Artificial
  synthetic construct
gggagagggauccagcugacgaguacugaauaccagccgaaaggcccuuggcagguuuggugaaacgccuucggcguccuggauuagaacuuc

SEQ ID NO: 6 - RNA, 93 nt, Artificial
  synthetic construct
gggagagggauccagcugacgannnnnnaauaccagccgaaaggcccuuggcaggnnnnnngaaacgccuucggcguccuggauuagaacuuc